(19) United States
(12) Patent Application Publication
(10) Pub. No.: US 2015/0018606 A1
(43) Pub. Date: Jan. 15, 2015

(54) CATHETER ASSEMBLY

(71) Applicant: TERUMO KABUSHIKI KAISHA, Shibuya-ku (JP)

(72) Inventors: Shigeki ARIURA, Ebina-shi (JP); Masakatsu KAWAURA, Sunnyvale, CA (US)

(73) Assignee: TERUMO KABUSHIKI KAISHA, Shibuya-ku (JP)

(21) Appl. No.: 14/279,973

(22) Filed: May 16, 2014

Related U.S. Application Data
(63) Continuation of application No. PCT/JP2012/079775, filed on Nov. 16, 2012.

(30) Foreign Application Priority Data
Nov. 18, 2011 (JP) 2011-253123

(51) Int. Cl. A61B 19/00; A61M 25/01; A61M 25/00; A61B 17/02

(52) U.S. Cl. CPC A61B 19/24; A61B 17/0218; A61M 25/0102; A61M 25/0023; A61M 25/005; A61M 2025/0063; A61M 2210/1092
USPC 600/37

(57) ABSTRACT

The catheter assembly includes a catheter having a catheter body, and a curved stylet that is constituted of a linear body of higher rigidity than the catheter body and is inserted into the catheter, the catheter assembly being assembled by inserting the curved stylet into the catheter. The curved stylet has a curved section, a part of which is curved. A deformation section that has been deformed in a curved manner along the curved shape of the curved section is formed on the catheter body at a portion where the curved section is located in the assembled state. The catheter assembly is so configured that, when the catheter is inserted into the urethral lumen in the assembled state, the deformation section curves a portion of the urethral lumen that faces the deformation section.

### CROSS-REFERENCES TO RELATED APPLICATIONS

This application is a continuation of International Application No. PCT/JP2012/079775 filed on Nov. 16, 2012, and claims priority to Japanese Application No. 2011-253123 filed on Nov. 18, 2011, the entire content of both of which is incorporated herein by reference.

### TECHNICAL FIELD

The present invention generally relates to a catheter assembly.

### BACKGROUND DISCUSSION

In a patient suffering from urinary incontinence, particularly stress urinary incontinence, involuntary urination occurs due to an abdominal pressure exerted during a normal exercise or by laughing, coughing, sneezing or the like. This is attributable, for example, to loosening of the pelvic floor muscle, which is a muscle for supporting the urethra, caused by childbirth or the like.

For treatment of urinary incontinence, surgical therapy is effective. For instance, a tape-shaped implant called a “sling” is set indwelling in the body so as to support the urethra by the sling. An example is disclosed in Japanese Patent Application Publication No. 2010-99499. In order to put a sling indwelling in the body, an operator incises the vagina with a surgical knife, dissects a biological tissue between the urethra and the vagina, and provides communication between the dissected (excised) biological tissue and the exterior through an obturator foramen by using a puncture needle or the like. Then, in such a state, the sling is set indwelling in the dissected biological tissue in the body.

However, since the biological tissue between the urethra and the vagina is a very thin layer, an insufficient skill of the operator may lead to damage of the urethra during dissection of the biological tissue. In addition, where the dissected (excised) layer of the biological tissue is close to the vagina side, the sling set indwelling may be located excessively close to the vagina side. In such a case, with the lapse of time after the surgery, the sling may come to break through the vaginal wall, to be exposed to the inside of the vagina.

### SUMMARY

According to one aspect, a catheter assembly includes a catheter comprised of a catheter body possessing at least one lumen, and a stylet comprised of a linear body positionable in the lumen in the catheter body in an assembled state of the catheter assembly. The stylet includes a curved section that is curved, and the linear body of the stylet, inclusive of the curved section, possesses a rigidity greater than the rigidity of the catheter body. The catheter body includes a deformation section that is deformable so that when the stylet is positioned in the lumen with the curved section of the stylet located in the lumen in the assembled state of the catheter assembly the deformation section of the catheter body deforms to a curved shape caused by the curved section of the stylet, and when the stylet is removed from the lumen the deformation section of the catheter body changes to a shape different from the curved shape. When the catheter in the assembled state is positioned in one of two living-body lumens adjacent to each other with biological tissue between the two living-body lumens, the deformation section of the catheter body which possess the curved shape curves a portion of the one living-body lumen facing the deformation section.

The curved section of the stylet preferably includes a first curved portion curved in one direction, and two second curved portions each curved in an opposite direction compared with the first curved portion, with one of the second curved portions being located immediately on a distal side of the first curved portion and the other second curved portion and immediately on a proximal side of the first curved portion.

The stylet preferably includes another section different from the curved section, with the other section of the stylet possessing a rectilinear shape in a natural state when no force is applied to the stylet, and where the distance between a straight line and a central portion of the first curved portion in a longitudinal direction of the first curved portion is not more than 10 mm, wherein the straight line interconnects both end sides of the stylet, and the curved section possessing a length along the straight line that is not more than four times the distance.

The curved section is preferably provided at an intermediate portion of the stylet in a longitudinal direction of the stylet so that a portion of the stylet extends from a proximal end of the curved section to a proximal end of the stylet and so that a portion of the stylet extends from a distal end of the curved section to a distal end of the stylet. The stylet possesses an outer diametrical size throughout the curved section that is greater than the outer diametrical size throughout the portion of the stylet on the distal side of the curved section and throughout the portion of the stylet on the proximal side of the curved section.

The curved section can be provided at an intermediate portion in a longitudinal direction of the stylet, with a portion of the stylet that is on a distal side of the curved section possessing a tapered shape where the outside diameter of the stylet gradually decreases along a distal direction.

The stylet can include a marker identifying the range over which the deformation section extends.

The marker can be a luminous element that emits light.

Alternatively, the marker can include a projecting portion that projects from an outer circumferential portion of the stylet.

Alternatively, the marker is a portion having an imaging property.

The stylet can also include a portion that identifies the direction of curvature of the curved section.

Restriction means for restricting rotation of the stylet about an axis of the catheter body in the assembled state can be provided.

The outer surface of the stylet can be a reduced friction outer surface treated by to reduce friction between the outer surface of the stylet and the catheter at the time of obtaining the assembled state.

The catheter body can include a longitudinally extending reinforcement member that prevents extension and contraction of the catheter body in the longitudinal direction.

The catheter body can also include a longitudinally extending inner layer and a longitudinally extending outer layer, with the a longitudinally extending reinforcement member disposed between the a longitudinally extending inner layer and the a longitudinally extending outer layer. The reinforcement member can be in the form of a spirally wound coil.

The reinforcement member can be configured to exhibit a lower winding density on the distal side than on the proximal side.

The deformation section of the catheter body can be located at a portion of the catheter body at which the winding density is low.

The stylet can be inserted and pulled out, and the catheter assembly can further comprise a correcting pipe insertable into the lumen together with the stylet in the inserted state to forcibly deform the curved section into a rectilinear shape.

The catheter body has two lumens, and the stylet is insertable into one of the two lumens, whereas a high-rigidity stylet having higher rigidity than the stylet and forcibly deforming the curved section in the assembled state is insertable into the other lumen.

The high-rigidity stylet can also include a luminous element that emits light, at its portion corresponding to the curved section in the assembled state.

According to another aspect, a catheter assembly includes a catheter comprised of a catheter body that includes a lumen, a stylet including a curved section that is curved in a natural state in which no force is applied to the curved section, the curved section of the stylet possessing a rigidity, and at least a portion of the catheter possessing a rigidity that is less than the rigidity of the curved section of the stylet. The curved section of the stylet which is curved being positioned inside the lumen in the catheter body so that the portion of the catheter body which possesses a rigidity less than the rigidity of the curved section surrounds the curved section of the stylet and is curved in a manner matching the curvature of the curved section of the stylet so that the portion of the catheter body is a curved portion. The rigidity of the curved section of the stylet causes, when the catheter is positioned in one of two living-body lumens adjacent to each other with biological tissue between the two living-body lumens, the curved portion of the catheter body to spread a portion of the biological tissue between the two living-body lumens to space the two living-body lumens further away from each other as compared to before positioning the catheter in the one of two living-body lumens.

The stylet is preferably a rectilinear shape, and the curved section of the catheter is forcibly deformed into a rectilinear shape along the stylet in the assembled state.

The catheter can include a radiopaque section which is flexible and which is disposed on the catheter body along the longitudinal direction of the catheter body.

The stylet can have a radiopaque section which is flexible and which is disposed on the stylet along the longitudinal direction of the stylet.

In accordance with another aspect, a method includes inserting a catheter into one of two living-body lumens positioned adjacent to each other with biological tissue between the two living-body lumens, wherein the insertion of the catheter into the one living-body lumen is performed to position at least a part of the catheter in the one living-body lumen. The method also comprises changing a shape of a portion of the catheter that is positioned in the one living-body lumen by curving the portion of the catheter in a direction away from the other living-body lumen so that the curving portion of the catheter causes a portion of the biological tissue facing the curving portion to be pulled away from the other living-body lumen.

One of the two living-body lumens is a urethral lumen, and the other living-body lumen is a vaginal cavity, and the catheter assembly is for use in therapy of a disease in pelvic viscera.

The catheter assembly disclosed here is configured so that at the time of applying a surgical treatment to a biological tissue between two living-body lumens, the treatment can be carried out rather easily and assuredly.

For instance, where the catheter assembly is used for therapy of female urinary incontinence, the catheter assembly is inserted into a urethral lumen in an assembled state.

In the therapy or treatment of urinary incontinence, a part of the biological tissue located between the urethral lumen and the vaginal cavity is dissected, and the thus dissected or excised part and the exterior of the body are caused to communicate with each other, through a puncture hole formed by puncture or the like, for example. Then, an implant for supporting the urethra is set indwelling in the puncture hole in the biological tissue.

Since the catheter is formed with the deformation section, when the catheter assembly in the assembled state is inserted into the urethral lumen, a part of the biological tissue that faces the deformation section is spread by the deformation section in such direction as that the urethral lumen and the vaginal cavity are spaced away from each other. In addition, the thus spread portion is spread to an extent sufficient for execution of the dissection and the puncture. The catheter assembly disclosed here thus helps ensure that that when a surgical treatment such as dissection and puncture is applied to the spread portion, the treatment can be carried out relatively easily and assuredly.

### BRIEF DESCRIPTION OF DRAWINGS

FIG. 1 shows side views illustrating a first embodiment of the catheter assembly disclosed here, where (a) is a side view and an auxiliary cross-sectional view of a catheter, and (b) and (c) are side views of a stylet.

FIGS. 2(a) and 2(b) are partial longitudinal cross-sectional views sequentially illustrating a method of using the catheter assembly shown in FIG. 1.

FIGS. 3(a) and 3(b) are partial longitudinal cross-sectional views sequentially illustrating the method of using the catheter assembly shown in FIG. 1.

FIGS. 4(a) and 4(b) are partial longitudinal cross-sectional views sequentially illustrating the method of using the catheter assembly shown in FIG. 1.

FIG. 5 is a partial longitudinal cross-sectional view illustrating the method of using the catheter assembly show in FIG. 1.

FIG. 6 is a cross-sectional view taken along the section line 6-6 in FIG. 5.

FIG. 7 is a partial longitudinal cross-sectional view illustrating a state in use of a catheter assembly according to a second embodiment representing another example of the catheter assembly disclosed here.

FIG. 8 is a partial longitudinal cross-sectional view illustrating a state in use of a catheter assembly according to a third embodiment representing another example of the catheter assembly disclosed here.

FIG. 9 is a cross-sectional view as seen from the direction of the arrow 9 in FIG. 8.

FIG. 10 is a partial longitudinal cross-sectional view illustrating a state in use of a catheter assembly according to a fourth embodiment representing another example of the catheter assembly disclosed here.

FIG. 11 is a cross-sectional view as seen from the direction of the arrow 11 in FIG. 10.

FIG. 12 is a partial longitudinal sectional view illustrating a state in use of a catheter assembly according to a fifth embodiment representing another example of the catheter assembly disclosed here.

FIG. 13 is a cross-sectional view as seen from the direction of the arrow 13 in FIG. 12.

FIGS. 14(a) and 14(b) illustrate a sixth embodiment of the catheter assembly disclosed here, where FIG. 14(a) is a partial longitudinal cross-sectional side view and an auxiliary plan view of a catheter, and FIG. 14(b) is a side view and an auxiliary sectional view of a stylet).

FIG. 15 is a side view showing the vicinity of a proximal portion of the catheter assembly of FIG. 14 in its assembled state.

FIG. 16 is a side view of a stylet in a catheter assembly according to a another embodiment.

FIG. 17 is a partial longitudinal cross-sectional view illustrating a state in use of a catheter assembly according to another embodiment.

FIG. 18 is a partial longitudinal cross-sectional view illustrating a state in use of a catheter assembly according to a another embodiment.

FIG. 19 is a partial longitudinal cross-sectional view illustrating a state in use of a catheter assembly according to a another embodiment.

FIGS. 20(a) and 20(b) are side views illustrating an eleventh embodiment of the catheter assembly disclosed here, where FIG. 20(a) is a side view illustrating a state in which a curved section of a stylet is covered by a correcting pipe, and FIG. 20(b) is a side view illustrating a state in which the correcting pipe has retracted from the curved section of the stylet.

FIGS. 21(a) and 21(b) are side views illustrating a twelfth embodiment of the catheter assembly disclosed here, where FIG. 21(a) is a side view and an auxiliary cross-sectional view illustrating a state in which a high-rigidity stylet is inserted in a catheter, and FIG. 21(b) is a side view and an auxiliary cross-sectional view illustrating a state in which the high-rigidity stylet has been pulled out of the catheter.

FIG. 22 is a partial longitudinal cross-sectional view illustrating a state in use of a catheter assembly according to according to a further embodiment representing another example of the catheter assembly disclosed here.

FIGS. 23(a)-23(d) are side views as seen from the direction of the arrow 23 in FIG. 22.

FIGS. 24(a) and 24(b) are partial longitudinal cross-sectional views illustrating a state in use of a catheter assembly according to an additional embodiment representing another example of the catheter assembly disclosed here.

FIG. 25 is a partial longitudinal cross-sectional view illustrating a catheter assembly according to another embodiment representing another example of the catheter assembly disclosed here.

FIG. 26 is a cross-sectional view taken along the section line 26-26 in FIG. 25.

FIG. 27 is a side view of a catheter in a catheter assembly according to a further embodiment representing another example of the catheter assembly disclosed here.

FIG. 28 is a magnified side view of a radiopaque section possessed by the catheter shown in FIG. 27.

FIG. 29 is a partial longitudinal cross-sectional view illustrating a state in use of the catheter assembly according to another embodiment representing another example of the catheter assembly disclosed here.

FIGS. 30(a) and 30(b) are side views illustrating a further embodiment of the catheter assembly, where FIG. 30(a) is a side view illustrating a state in which a curved section of a stylet is covered by a correcting pipe, and FIG. 30(b) is a side view illustrating a state in which the correcting pipe is retracted from the curved section of the stylet.

FIGS. 31(a)-31(c) are side views illustrating a further embodiment of the catheter assembly disclosed here, where FIG. 31(a) is a side view illustrating a state in which a high-rigidity stylet is inserted in a catheter, FIG. 31(b) is a side view illustrating a state in which the high-rigidity stylet has been pulled out of the catheter, and FIG. 31(c) is a side view illustrating a state in which an endoscope is inserted in the catheter.

### DETAILED DESCRIPTION

Set forth below. With reference to the accompanying drawing figures, is a detailed description of embodiments of a catheter assembly representing examples of the inventive catheter assembly disclosed here

### First Embodiment

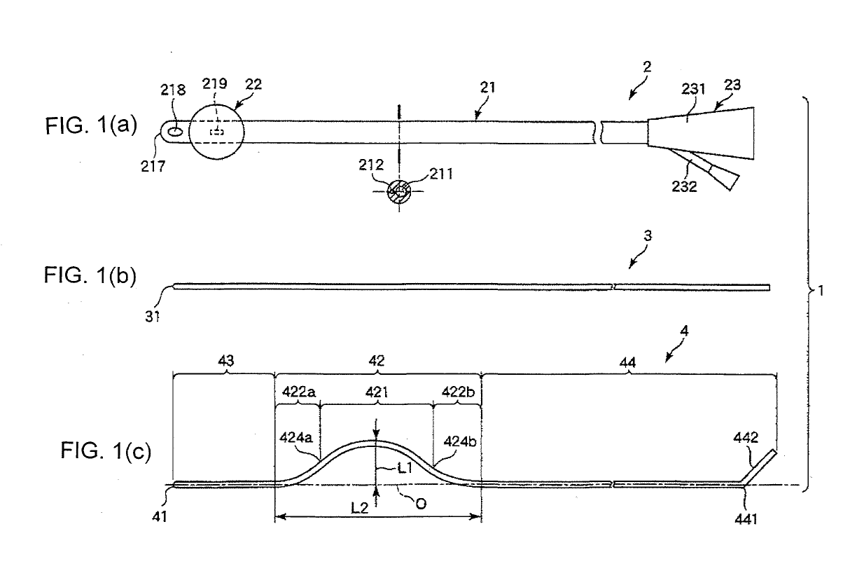

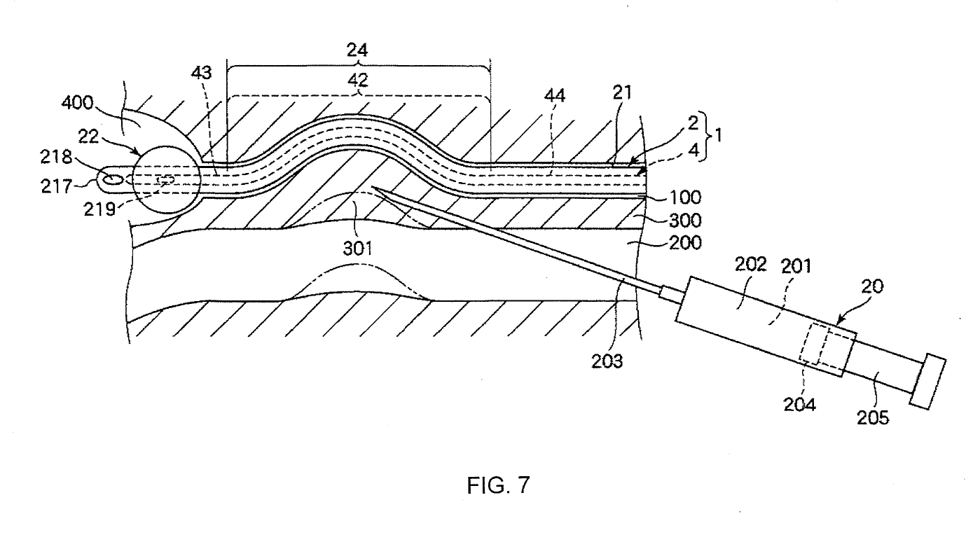

FIGS. 1-6 illustrate a first embodiment of the catheter assembly disclosed here. In the following description, the right side in FIGS. 1 to 5 (and in FIGS. 7, 8, 10, 12, 24 to 25, and 27 to 31, as well) are referred to as the “proximal (end),” and the left side is referred to as the “distal (end),” for convenience of description.

The catheter assembly 1 shown in FIG. 1 is a urethral catheter to be used by being inserted into a urethral lumen (a living-body lumen) 100 during treatment of female urinary incontinence, which is one of treatments of diseases in pelvic viscera, specifically at the time of burying an implant (an instrument to be set indwelling in a living body) in a living body.

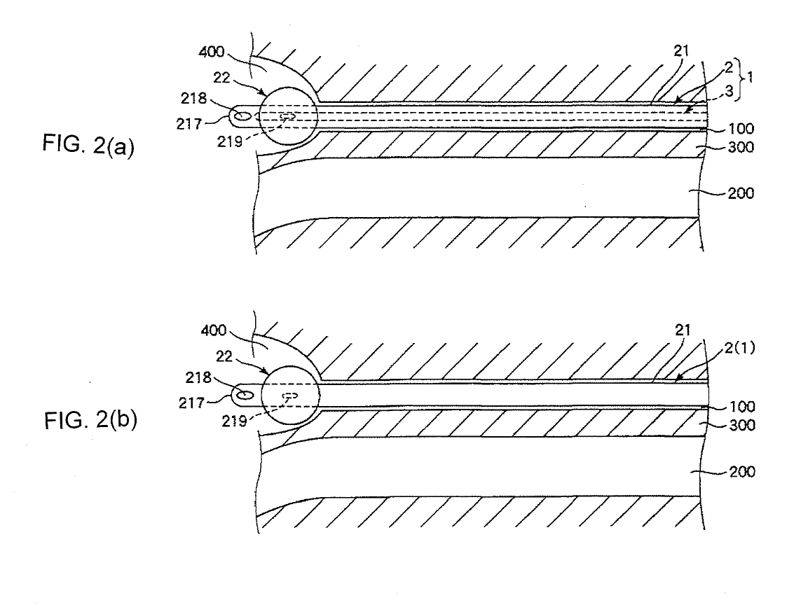

The urethral lumen 100 is adjacent to a vaginal cavity (another living-body lumen) 200, with a biological tissue 300 located between the urethral lumen 100 and the vaginal cavity (see FIGS. 2 to 6). The biological tissue 300 includes a wall section (urethral wall) defining the urethral lumen 100 and a wall section (vaginal wall) defining the vaginal cavity 200. In addition, the thickness of the biological tissue 300, which differs from individual to individual, is generally about 5 to 20 mm in the case of female adults.

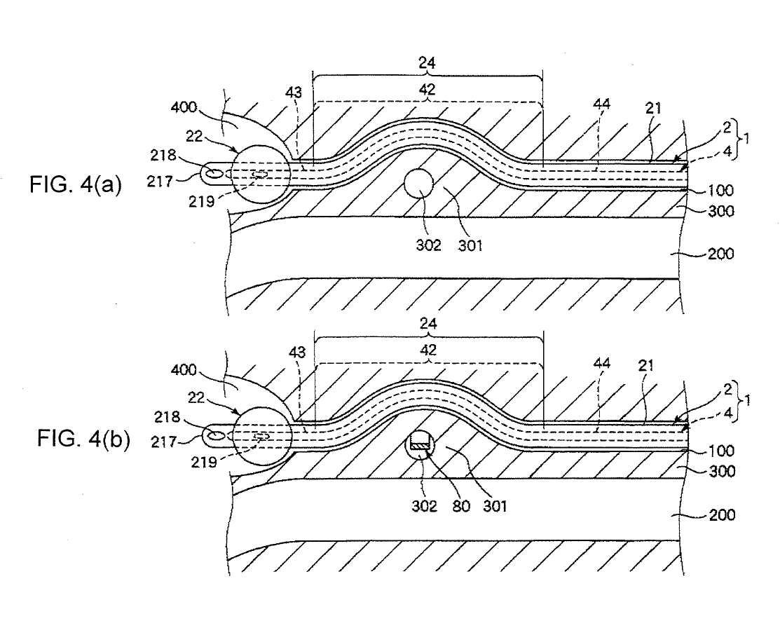

The implant is an instrument which, for treatment of female urinary incontinence, is buried in the biological tissue 300 to support the urethra in the manner of pulling the urethra away from the vaginal wall (see FIGS. 4(f), 5(g), and 6). In this way, the urethra is supported by the implant, and involuntary urination is prevented.

Examples of the implant include a flexible long (elongated) body. In the present embodiment, the implant is composed of a belt 80. This belt 80 is called a “sling.”

In addition, the dimensions of the belt 80 are not particularly limited but are set as required. Preferably, however, the width of the implant is about 3 to 15 mm, and the thickness of the implant is about 0.2 to 2 mm.

The material constituting the belt 80 is not specifically restricted. For instance, various biocompatible resin materials and the like can be used as the material for the belt 80.

The implant is composed of a single belt 80 in this embodiment, but this is not a limitation. For example, the implant may be composed of a plurality of belts 80.

In addition, the implant is not restricted to the belt 80. For example, other flexible long (elongated) bodies such as threads and strings can be used. In the case where a thread or string is used as the implant and the thread or string is circular in cross-sectional shape, the diameter is preferably about 0.2 to 5 mm.

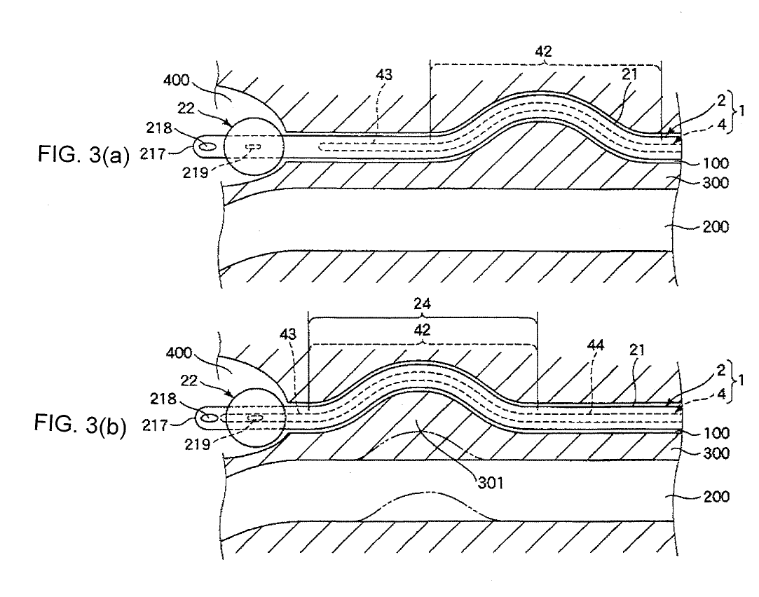

As shown in FIG. 1, a catheter assembly 1 includes a catheter 2, a rectilinear stylet (first stylet) 3, and a curved stylet (second stylet) 4. The rectilinear stylet 3 and the curved stylet 4 are configured to be selectively inserted into the catheter 2. As a result, the catheter assembly 1 assumes a first assembled state (see FIG. 2(a)) in which it is assembled by inserting the rectilinear stylet 3 into the catheter 2 and a second assembled state (see FIGS. 3 and 4) in which it is assembled by inserting the curved stylet 4 into the catheter 2. The configuration of each of the sections or portions will be described below.

As shown in FIG. 1(a), the catheter 2 is a balloon catheter that has a catheter body 21, a balloon 22 disposed at a distal portion of the catheter body 21 and configured to be inflated and deflated, and a hub 23 disposed at a proximal portion of the catheter body 21.

The catheter body 21 is composed of a tube (tubular) body that is flexible and is substantially rectilinear in a natural state when no external force is exerted on catheter body 21. The tube body (catheter body 21) may be either monolayer or multilayer in form; in this embodiment, the tube body is in a monolayer form. In this case, the material constituting the tube body is not particularly limited. Examples of the material which can be used here include various thermoplastic and thermosetting resins, such as polyolefin resins, polyamide resins, polyurethane resins, and polyimide resins. Specific examples of the applicable material include polyolefins such as polyethylene, polypropylene, ethylene-propylene copolymers, ethylene-vinyl acetate copolymers (EVA), etc.; polyvinyl chloride; polyesters such as polyethylene terephthalate (PET), polybutylene terephthalate (PBT), etc.; polyurethane; polyamides; polyimides; polystyrene resins; fluoro-resins; and various thermoplastic elastomers based on styrene, polyolefin, polyvinyl chloride, polyurethane, polyester, polyamide, polybutadiene, fluoro-rubber or the like.

The catheter body 21 has lumens 211 and 212. The lumen 211 is greater than the lumen 212 in cross-sectional size.

The rectilinear stylet 3 or the curved stylet 4 is selectively inserted in the lumen 211. This lumen 211 opens at the distal portion of the catheter body 21, forming a side hole 218. That is, the lumen 211 opens to the outside of the catheter body 21 at the side hole 218. The number of side hole(s) 218 here is not particularly limited; for example, one side hole or a plurality of side holes may be formed.

The lumen 212 is disposed at a position different from the position of the lumen 211. That is, the two lumens 212, 211 are separate from one another. The lumen 212 constitutes a flow path through which a working fluid for inflation and deflation of the balloon 22 passes. In addition, the lumen 212 opens at a distal portion of the catheter body 21 in a position on the proximal side of the side hole 218, forming a side hole 219. That is, the lumen 212 opens to the outside of the catheter body 21 at the side hole 219. The working fluid can be fed into and out of the balloon 22 via the side hole 219, so that the balloon 22 is inflated and deflated.

In addition, the distal end 217 of the catheter body 21 is rounded. This helps ensure that when the catheter assembly 1 is inserted into the urethral lumen 100, the biological tissue 300 can be prevented from being damaged by the distal end 217. Consequently, insertion of the catheter assembly 1 into the urethral lumen 100 can be carried out safely.

The balloon 22 is composed of a membrane possessing a tubular shape, and is set in place by gas-tight attachment of a proximal portion and a distal portion of the balloon 22 onto an outer circumferential surface of the catheter body 21. The side hole 219 opens into the inside of the balloon 22. That is the side hole 219 is located in the balloon 22. When supplied with a working fluid, the balloon 22 is inflated into a spherical shape. Upon inflation of the balloon 22 in the condition where the catheter assembly 1 is inserted in the urethral lumen 100, the balloon 22 can rather easily engage with an inner wall of a bladder 400. This helps ensure that the catheter assembly 1 can be securely prevented from being pulled out of the urethral lumen 100 in an unintentional manner. The method for attaching the balloon 22 onto the catheter body 21 is not specifically restricted. For example, a method by fusion bonding (thermal fusion bonding, high-frequency fusion bonding, ultrasonic fusion bonding, etc.), a method by adhesion (adhesion by use of an adhesive or a solvent) or the like method can be used.

The material constituting the balloon 22 is not particularly limited. Examples of the material applicable here include: polyester resins such as polyethylene terephthalate, polyethylene naphthalate, polybutylene terephthalate, polybutylene naphthalate, etc. and polyester elastomer containing such a polyester resin; olefin resins such as polyethylene, polypropylene, etc. and cross-linked products of such olefin resins (especially, products cross-linked by irradiation with electron beams); polyamide resins such as nylon 11, nylon 12, nylon 610, etc. and polyamide elastomer containing such a polyamide resin; polyurethane resin; ethylene-vinyl acetate copolymers and cross-linked products thereof; and polymer blends, polymer alloys and the like containing at least one of these resin materials.

The working fluid for inflation and deflation of the balloon 22 is not specifically restricted. Examples of the working fluid applicable here include liquids such as physiological saline solution, etc. and gases such as air, carbon dioxide gas, etc.

The hub 23 includes a tubular main body section 231, and a tubular branch port 232 branching from an intermediate portion in the longitudinal direction of the main body section 231.

The main body section 231 communicates with the lumen 211 in the catheter body 21. The rectilinear stylet 3 and the curved stylet 4 can be inserted into the lumen 211 via the main body section 231.

The branch port 232 communicates with the lumen 212 in the catheter body 21. A syringe, for example, can be connected to the branch port 232. By operating the syringe, the working fluid can flow distally and proximally within the lumen 212.

The material constituting the hub 23 is not particularly limited. Examples of the applicable material include resin materials such as polyvinyl chloride, polyethylene, polypropylene, polycarbonate, polymethyl methacrylate, acrylonitrile-styrene-butadiene copolymer, etc. and various metallic materials.

The catheter 2 in this embodiment is a balloon catheter having the balloon 22, but the catheter is not limited in this regard. A catheter in which such balloon 22 is omitted may also be used. In the case where the catheter 2 is a catheter in which the balloon 22 is omitted, the catheter body 21 can be a catheter body having only one lumen 211.

As shown in FIG. 1(b), the rectilinear stylet 3 is composed of a linear body which has a substantially rectilinear shape in a natural state. The natural state refers to the state in which no force (external or internal) is applied to the stylet. In addition, the outside diameter of the rectilinear stylet 3 is constant along the longitudinal direction of the stylet. The rectilinear stylet 3 is preferably circular in cross-sectional shape, but the stylet is not limited in this regard. The rectilinear stylet 3 may be elliptic or polygonal in cross-sectional shape.

The distal end 31 of the rectilinear stylet 3 is rounded. This helps ensure that when the rectilinear stylet 3 is inserted into the lumen 211 of the catheter body 21 to obtain the first assembled state, the distal end 31 is prevented from being caught on an inner circumferential portion of the catheter body 21. Therefore, the inserting operation can be carried out rather easily.

In addition, the rigidity of the rectilinear stylet 3 is greater than the rigidity of the catheter body 21. This helps ensure that when the catheter assembly 1 in the first assembled state is inserted into the urethral lumen 100 (see FIG. 2), the catheter body 21 can be prevented from unintentionally curving or bending during the insertion process. Therefore, the inserting operation can be performed rather easily and assuredly. The method for setting the rigidity of the rectilinear stylet 3 higher than the rigidity of the catheter body 21 is not specifically restricted. Examples of the applicable method include a method in which the material constituting the rectilinear stylet 3 is selected as required, and a method in which the diametrical size (thickness) of the rectilinear stylet 3 is selected as necessary.

The material or materials constituting the rectilinear stylet 3 and the curved stylet 4 are not particularly limited. For example, the material(s) may be stainless steel.

As shown in FIG. 1(c), the curved stylet 4 is composed of a linear body of higher rigidity than the catheter body 21, like the rectilinear stylet 3. The outside diameter of the curved stylet 4 is constant along the longitudinal direction. The outside diameter of the curved stylet 4 may be the same as or different from the outside diameter of the rectilinear stylet 3. In addition, the rectilinear stylet 3 is preferably circular in cross-sectional shape, but this is not restrictive. The rectilinear stylet 3 may be elliptic or polygonal in cross-sectional shape.

The distal end 41 of the curved stylet 4 is rounded. This helps ensure that, when the curved stylet 4 is inserted into the lumen 211 in the catheter body 21 to obtain the second assembled state, the distal end 41 is prevented from being caught on an inner circumferential portion of the catheter body 21. Therefore, the inserting operation can be conducted rather easily.

The curved stylet 4 has a curved section 42 which is curved in a natural state, at an intermediate portion in the longitudinal direction thereof, specifically, at its portion on the distal side. The natural state refers to the state in which no force (external or internal) is applied to the curved section of the curved stylet. The curved shape of the curved section 42 is substantially maintained even in the second assembled state (see FIGS. 3(b), 4(a) and 4(b)).

As shown in FIG. 1(c), the curved section 42 can be divided into a first portion 421, and second portions 422a and 422b which are continuously formed respectively immediately on the distal side of and immediately on the proximal side of the first portion 421. The first portion 421 is so curved as to be “projected (protuberant)”, or so as to project, in one direction, specifically, toward the upper side in FIG. 1(c). The second portions 422a and 422b are so curved as to be “projected (protuberant)”, or so as to project, in the opposite direction as compared with the first portion 421, specifically, toward the lower side in FIG. 1(c). In addition, a boundary portion between the first portion 421 and the second portion 422a and a boundary portion between the first portion 421 and the second portion 422b are “inflection points” where the direction of curvature changes. In the illustrated embodiment, the distal-most end of the curved section 42 at which the curved section 42 transitions to the adjoining section of the stylet (the rectilinear section 43) is proximally spaced from the distal-most end of the stylet 4. Similarly, the proximal-most end of the curved section 42 at which the curved section 42 transitions to the adjoining section of the stylet (the rectilinear section 44) is distally spaced from the proximal-most end of the stylet 4.

As shown in FIGS. 3(b), 4(a) and 4(b), in the second assembled state, the portion of the catheter body 21 at which the curved section 42 is located is shaped along the curved shape of the curved section 42. Consequently, the catheter body 21 exhibits a deformation section 24 deformed in a curved manner substantially like the curved section 42 is formed. The central axis of the curved deformation section 24 is curved.

In the catheter assembly 1 (catheter 2) in the second assembled state in which the catheter assembly is inserted in the urethral lumen 100, the deformation section 24 can curve a portion of the urethral lumen 100 that faces the deformation section 24, whereby a portion of the biological tissue 300 that faces the deformation section 24 can be pulled toward the deformation section 24 side (the upper side in FIGS. 3(b), 4(a) and 4(b)). In other words, a vertex of the deformation section 24 is disposed in such a direction as to be spaced away from the living-body lumen (the vaginal cavity 200) in which the catheter assembly 1 is not inserted, of the two living-body lumens (the urethral lumen 100 and the vaginal cavity 200) adjacent to each other. The vertex of the deformation section 24 is spaced farther from the vaginal cavity 200 than other portions of the catheter assembly disposed proximally and distally of the vertex. This arrangement helps ensure that the above-mentioned portion is curved and spread in such direction that the urethral lumen 100 and the vaginal cavity 200 are spaced away from each other. Hereinafter, this spread portion will be referred to as “the expanded portion 301.” The expanded portion 301 is formed therein with a puncture hole 302, in which the belt 80 is set indwelling.

The biological tissue 300 is spread by the deformation section 24, normally by about one-and-a-half to three times the size of the biological tissue 300 before being spread.

In addition, assume a straight line O (shown in FIG. 1(c)) intersecting both end sides (ends) of the curved stylet 4 in the natural state. Then, of the curved section 42, the distance L₁ (shown in FIG. 1(c)) between the straight line O and a central portion (vertex portion) 423 in the longitudinal direction of the first portion 421 is not particularly limited; for example, the distance L₁ is preferably not more than 10 mm, more preferably in the range of 5 to 10 mm. The length L₂ (shown in FIG. 1(c)) of the curved section 42 along the straight line O is not specifically restricted; for example, the distance L₂ is preferably not more than four times the distance L₁, more preferably in the range of two to three times the distance L₁. The distance L₁ is greater than the diameter of the living-body lumen (e.g., urethral lumen 100) into which the curved stylet 4 is inserted during use of the catheter assembly.

When the portion of the biological tissue 300 that faces the deformation section 24 is pulled by the deformation section 24 toward the deformation section 24 side, as illustrated in the part indicated by two-dot chain line in FIG. 3(b), the portion defining the vaginal cavity 200 is also pulled in that direction. This results in a state where it seems that the expanded portion 301 would not be formed in the biological tissue 300. With the setting in the above-mentioned numerical value ranges, however, excessive pulling of the biological tissue 300 can be securely prevented, so that the expanded portion 301 can be formed rather assuredly.

In addition, the curved stylet 4 is configured so that sections other than the curved section 42, namely the distal-side section and the proximal-side section interconnected through the curved section 42, are rectilinear sections 43 and 44 which are rectilinear in shape in a natural state. The natural state refers to the state in which no force (external or internal) is applied to the distal-side section and the proximal-side section of the curved stylet. The rectilinear section 44 includes a bent section 441, a part (proximal portion) of which is bent, as in the configuration shown in FIG. 1. In this case, the section on the proximal side of the bent section 441 constitutes a projected section 442 which projects to the upper side in FIG. 1(c), namely, in the same direction as the direction of curvature of the first portion 421 of the curved section 42. The projected section 442 enables grasping of the direction of curvature of the curved section 42, particularly the direction of curvature of the first portion 421, in the second assembled state. Therefore, the biological tissue 300 can be securely deformed in the direction in which the biological tissue 300 is to be deformed by pulling. Consequently, the biological tissue 300 can be assuredly formed with the expanded portion 301.

Now, an example of the method of using the catheter assembly 1, or a procedure for burying the belt 80 into a living body in order to treat female urinary incontinence, will be described below referring to FIGS. 2 to 6.

[1] First, the rectilinear stylet 3 is inserted, starting from its distal end, into the lumen 211, through a main body section 231 of the hub 23 of the catheter 2, to put the catheter assembly 1 into the first assembled state. The catheter 2 thus exhibits a rectilinear shape. In this instance, the balloon 22 of the catheter 2 is not yet inflated but is in a deflated state. In addition, a syringe prefilled with physiological saline solution is prepared. The physiological saline solution is for use as a working fluid for actuating the balloon 22 of the catheter 2.

Then, as shown in FIG. 2(a), the catheter assembly 1 in the first assembled state (with the catheter possessing a rectilinear shape) is inserted into the urethral lumen 100, with the distal end of the catheter assembly 1 first being inserted into the urethral lumen. This insertion is conducted until the balloon 22 of the catheter 2 is located inside the bladder 400. Thereafter, the above-mentioned syringe is connected to the branch port 232 of the hub 23 of the catheter 2, and the syringe is operated to supply the physiological saline solution into the lumen 212 of the catheter 2. As a result, the balloon 22 is inflated, to engage the inner wall of the bladder 400 as shown in FIGS. 2(a) and 2(b). Consequently, the catheter assembly 1 is prevented from being unintentionally pulled out of the urethral lumen 100.

[2] Next, as shown in FIG. 2(b), the rectilinear stylet 3 is pulled out of the catheter 2. In this instance, the syringe may be disconnected from the branch port 232 of the hub 23 of the catheter 2.

[3] Subsequently, as shown in FIG. 3(a), the curved stylet 4 is gradually inserted, starting from the distal end of the curved stylet 4, into the lumen 211 through the main body section 231 of the hub 23 of the catheter 2 from which the rectilinear stylet 3 has been pulled out. The curved section 42 of the curved stylet 4 which is curved is thus positioned inside the lumen in the catheter body 21 so that the portion of the catheter body 21 which possesses a rigidity less than the rigidity of the curved section 42 of the curved stylet 4 surrounds the curved section of the stylet and is curved in a manner matching the curvature or curve of the curved section 42 of the curved stylet 4 so that the portion of the catheter body is a curved catheter body portion.

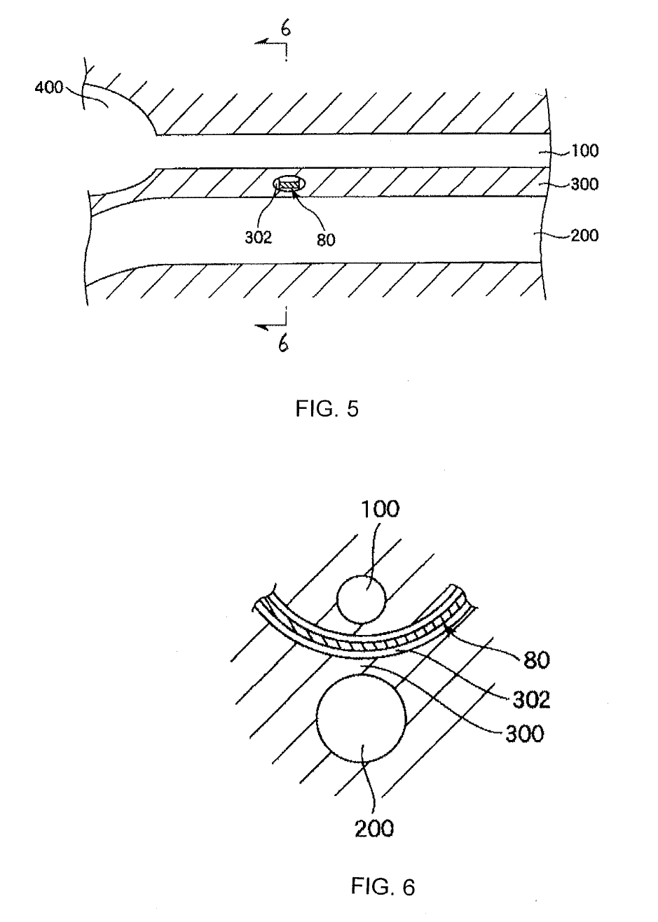

Consequently, as shown in FIG. 3(b), the catheter assembly 1 is put in the second assembled state. At this time, the catheter 2 is formed with the deformation section 24 (curved section) as described above. This deformation section 24 results because the curved stylet 4, inclusive of the curved section 42, possesses a rigidity greater than the rigidity of the catheter body 21. The deformed configuration of the deformation section 24 causes the portion of the biological tissue 300 that faces the deformation section 24 to be forcibly pulled (corrected) toward the upper side in FIG. 3(b) along the shape of the deformation section 24. That is, the portion of the biological tissue 300 in which the deformation section 24 is located takes on a shape or configuration that is the same as the deformation section 24. As a result, the expanded portion 301 of the biological tissue 300 which has been spread or moved in such direction that the urethral lumen 100 and the vaginal cavity 200 are spaced away from each other farther than before insertion of the curved stylet 4, namely, which has been enlarged in material thickness, is securely formed in the biological tissue 300.

[4] Next, as shown in FIG. 4(a), the expanded portion 301 is dissected or excised, and a puncture hole 302 for providing communication between the thus dissected (excised) expanded portion 301 and the exterior through a obturator foramen is formed by use of a puncture needle. The expanded portion 301 to which the dissection (excision) operation and the puncturing operation are to be applied has been expanded to an extent sufficient for these operations to be performed. Therefore, the catheter assembly 1 helps ensure that, at the time of applying the surgical treatment (in this embodiment, the dissection (excision) operation and the puncturing operation) to the expanded portion 301, the treatment can be carried out relatively easily and assuredly.

[5] Subsequently, as shown in FIG. 4(b), the belt 80 is passed through the puncture hole 302, by use of a guide wire, for example. The belt 80 is in a state of catching a urethral wall (the expanded portion 301), with both end portions of the belt protruding from the puncture hole 302 to the outside of the living body.

[6] Next, as shown in FIGS. 5 and 6, the catheter assembly 1 is pulled out of the urethral lumen 100. This pulling-out may be carried out while keeping the catheter assembly 1 in the second state. Alternatively, the curved stylet 4 may first be pulled out of the catheter 2, followed by pulling the catheter 2 out of the urethral lumen 100.

Then, both end portions of the belt 80 which have protruded to the exterior of the living body are each pulled with a predetermined force. This results in that the urethral wall is pulled in such a direction as to be spaced away from the vaginal wall, by the tension of the belt 80, and the urethra is supported by the belt 80.

Thereafter, an unnecessary portion of the belt 80 is cut away, followed by predetermined suture and the like, to complete the procedure.

In this embodiment, the catheter assembly 1 has been described referring to the case where an implant capable of being buried is buried (positioned) in a living body for treatment of female urinary incontinence, but this is not restrictive. The catheter assembly 1 can be applied to other uses as well.

### Second Embodiment

FIG. 7 illustrates a catheter assembly according to a second embodiment while the catheter assembly is in use. The following description focuses primarily on differences between this embodiment and the embodiment described above. Features in this second embodiment that are similar to features in the embodiment described above are identified by common reference numerals and a detailed description of such features is not repeated.

This embodiment is the same as the first embodiment, except for a difference in part of the procedure for treatment of female urinary incontinence.

As shown in FIG. 7, in this embodiment, in treatment of urinary incontinence, a liquid 201 is injected by a syringe 20 into a portion of the biological tissue 300 formed by the deformation section 24 of the catheter assembly 1 in a second assembled state and which constitutes the expanded portion 301 of the biological tissue 300.

The syringe 20 includes: a syringe outer tube 202; an injection needle 203 mounted to a mouth portion of the syringe outer tube 202; a gasket 204 slidably disposed within the syringe outer tube 202; and a plunger 205 operated to move the gasket 204. A space surrounded by the syringe outer tube 202 and the gasket 204 is filled with the liquid 201. The liquid 201 is not limited to a specific liquid. For example, a mixture of an anesthetic liquid and physiological saline solution can be used as the liquid 201. Furthermore, a medicine for enhancing a hemostatic effect may be mixed in the liquid 201.

Meanwhile, when a portion of the biological tissue 300 that faces the deformation section 24 is pulled by the deformation section 24 toward the deformation section 24 side, as shown in the part indicated by two-dot chain line in FIG. 7, a portion defining a vaginal cavity 200 is also pulled in the same direction. This results in a state where it seems that the expanded portion 301 would not be formed in the biological tissue 300. However, when the injection needle 203 of the syringe 20 is inserted via the vaginal cavity 200 and punctures a portion capable of constituting the expanded portion 301 of the biological tissue 300 and the liquid 201 is injected, the portion under consideration is expanded, whereby the biological tissue 300 can be securely prevented or restricted from being unwillingly deformed due to excessive pulling of the biological tissue 300. As a result, the expanded portion 301 can be formed more securely.

### Third Embodiment

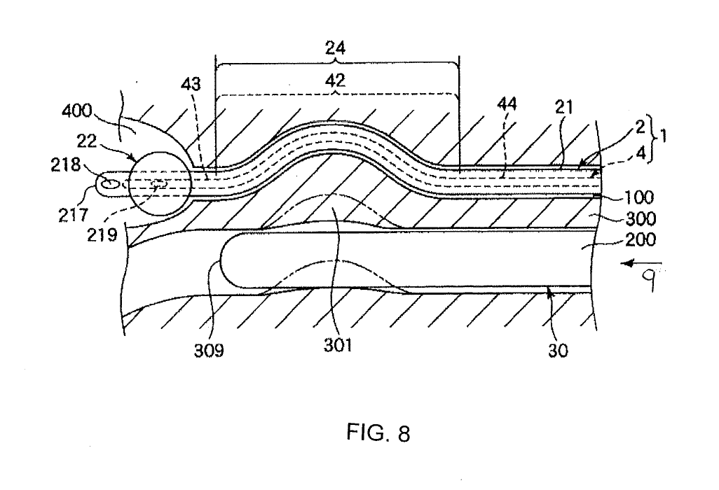

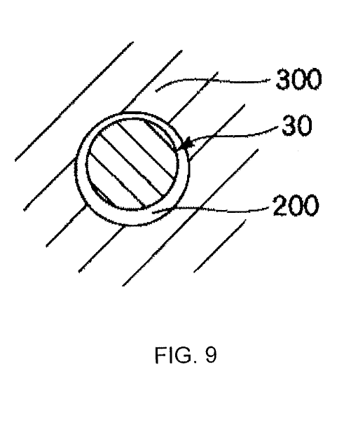

FIGS. 8 and 9 illustrate a catheter assembly according to a third embodiment while the catheter assembly is in use. The following description focuses primarily on differences between this embodiment and the embodiments described above. Features in this third embodiment that are similar to features in the embodiments described above are identified by common reference numerals and a detailed description of such features is not repeated.

This embodiment is the same as the first embodiment above, except for a difference in part of the procedure for treatment of female urinary incontinence.

As shown in FIGS. 8 and 9, in this embodiment, the treatment of urinary incontinence involves inserting a vaginal insertion instrument 30 into the vaginal cavity 200.

The vaginal insertion instrument 30 is composed of a hard bar-like body of which the outside diameter is constant along the longitudinal direction or extent. The outside diameter of the vaginal insertion instrument 30 is preferably approximately equal to the inside diameter of the vaginal cavity 200, in order that formation of a gap between the vaginal insertion instrument 30 and a vaginal wall is prevented as securely as possible.

As shown in FIG. 8, the distal end 309 of the vaginal insertion instrument 30 is rounded. In addition, as shown in FIG. 9, the vaginal insertion instrument 30 is circular in cross-sectional shape. Such a shape helps ensure that at the time of inserting the vaginal insertion instrument 30 into the vaginal cavity 200, a vaginal wall (biological tissue 300) defining the vaginal cavity 200 can be securely prevented from being damaged by the vaginal insertion instrument 30.

The material constituting the vaginal insertion instrument 30 is not particularly limited. Examples of materials include the same materials as those for the hub 23.

When a portion of the biological tissue 300 that faces a deformation section 24 is pulled by the deformation section 24 toward the deformation section 24 side, as shown in the part indicated by the two-dot chain line in FIG. 8, a portion defining the vaginal cavity 200 is also pulled in that same direction. This results in a state where it seems that an expanded portion 301 would not be formed in the biological tissue 300. With the vaginal insertion instrument 30 inserted in the vaginal cavity 200, however, unwilling deformation of the biological tissue 300 due to excessive pulling of the biological tissue 300 can be prevented or restricted more assuredly. Consequently, the expanded portion 301 can be formed in a further assured manner.

### Fourth Embodiment

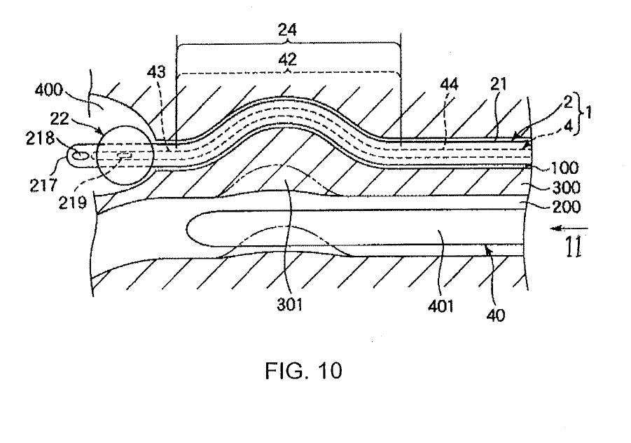

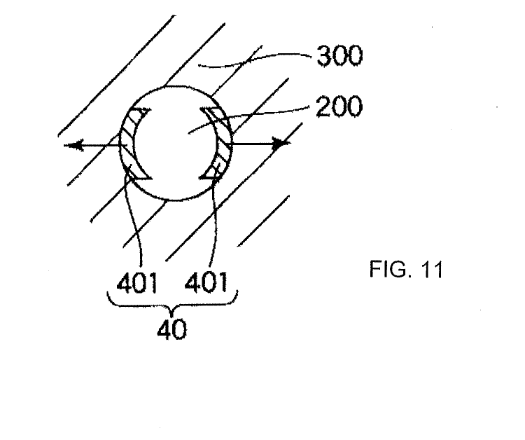

FIGS. 10 and 11 illustrate a catheter assembly according to a fourth embodiment while the catheter assembly is in use. The following description focuses primarily on differences between this embodiment and the embodiments described above. Features in this fourth embodiment that are similar to features in the embodiments described above are identified by common reference numerals and a detailed description of such features is not repeated.

This embodiment is the same as the first embodiment above, except for a difference in part of the procedure for treatment of female urinary incontinence.

As shown in FIGS. 10 and 11, the treatment of urinary incontinence involves inserting a colposcope 40 into a vaginal cavity 200.

The colposcope 40 has a pair of tongue pieces 401 configured to approach each other and move away from each other. The tongue pieces 401 are so configured that the spacing between the tongue pieces can be maintained.

Meanwhile, when a portion of a biological tissue 300 that faces a deformation section 24 is pulled by the deformation section 24 toward the deformation section 24 side, as shown in the part indicated by two-dot chain line in FIG. 10, a portion defining the vaginal cavity 200 is also pulled in that direction. This results in a state where it seems that an expanded portion 301 would not be formed in the biological tissue 300. However, when the colposcope 40 is inserted in the vaginal cavity 200 and the tongue pieces 401 are spaced away from each other (see FIG. 11), unwilling or undesired deformation of the biological tissue 300 due to excessive pulling of the biological tissue 300 can be prevented or restricted more securely. Consequently, the expanded portion 301 can be formed further assuredly.

### Fifth Embodiment

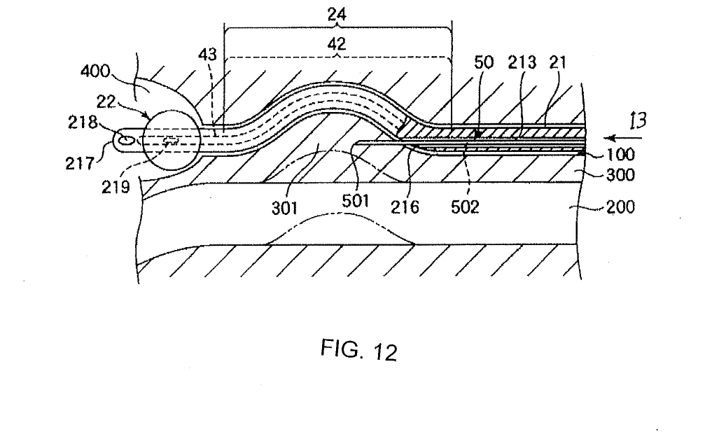

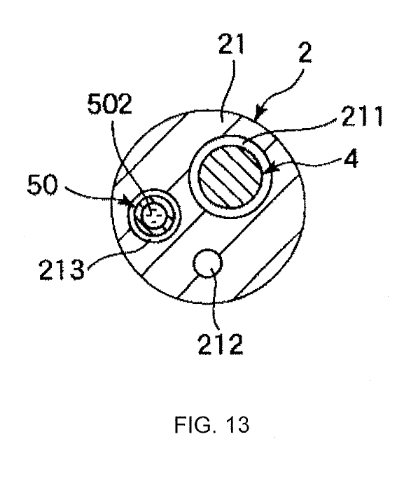

FIGS. 12 and 13 illustrate a catheter assembly according to a fifth embodiment while the catheter assembly is in use. The following description focuses primarily on differences between this embodiment and the embodiments described above. Features in this fifth embodiment that are similar to features in the embodiments described above are identified by common reference numerals and a detailed description of such features is not repeated.

This embodiment is the same as the first embodiment above, except for a difference in the configuration of the catheter.

As shown in FIGS. 12 and 13, in this embodiment, a catheter 2 further has a lumen 213, in addition to lumens 211 and 212. This lumen 213 forms a side hole 216 opening in a portion corresponding to a second portion 422b of a deformation section 24.

A long (elongated) tubular or hollow needle 50 having a sharp needle tip 501 is positioned in and passes through the lumen 213. When the tubular needle 50 is inserted and passed in the lumen 313, the needle tip 501 protrudes distally from the side hole 216. This helps ensure that a portion of a biological tissue 300 which will be an expanded portion 301 is punctured by the needle tip 501. Then, in the punctured state, a liquid 502 can be inserted into the portion under consideration, through the tubular needle 50. The liquid 502 is not restricted to a specific liquid. For example, a mixture of an anesthetic liquid and physiological saline solution can be used as the liquid 502. Furthermore, a medicine for enhancing a hemostatic effect may be mixed in the liquid 502.

Meanwhile, when a portion of the biological tissue 300 that faces the deformation section 24 is pulled by the deformation section 24 toward the deformation section 24 side, as shown in the part indicated by two-dot chain line in FIG. 12, a portion defining a vaginal cavity 200 is also pulled in that direction. This results in a state where it seems that an expanded portion 301 would not be formed in the biological tissue 300. However, when the tubular needle 50 inserted and passed in the lumen 213 punctures a portion of the biological tissue 300 that can constitute the expanded portion 301 and the liquid 502 is injected, the portion is expanded. This helps ensure that unwilling deformation of the biological tissue 300 due to excessive pulling of the biological tissue 300 can be prevented or restricted more securely. Consequently, the expanded portion 301 can be formed more assuredly.

### Sixth Embodiment

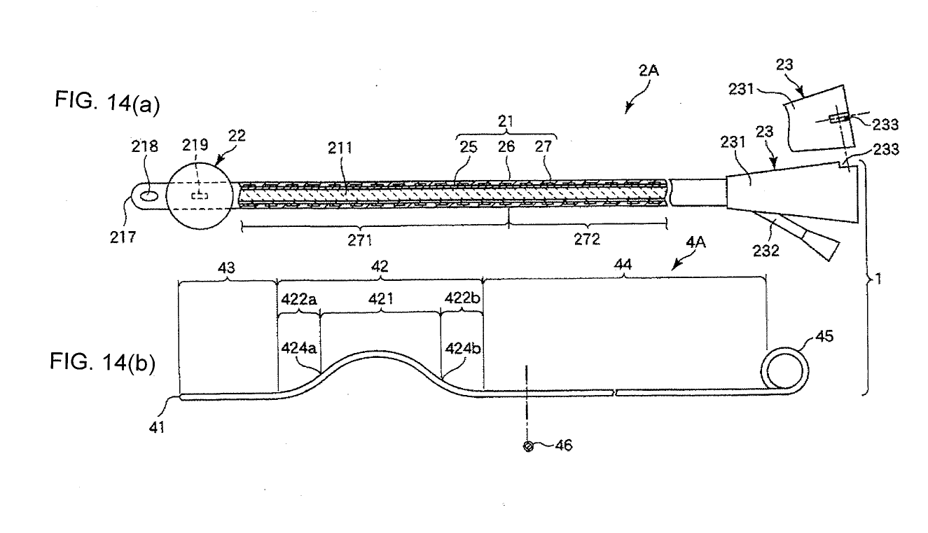

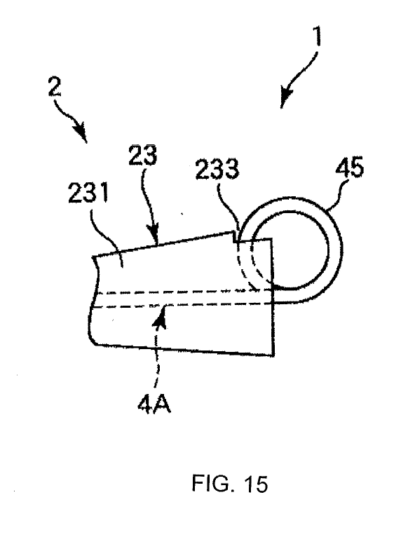

FIGS. 14 and 15 illustrate a catheter assembly according to a sixth embodiment while the catheter assembly is in use. The following description focuses primarily on differences between this embodiment and the embodiments described above. Features in this sixth embodiment that are similar to features in the embodiments described above are identified by common reference numerals and a detailed description of such features is not repeated.

This sixth embodiment is the same as the first embodiment above, except for differences in the configurations of a catheter and a curved stylet.

As shown in FIG. 14(a), in this embodiment, a catheter body 21 of a catheter 2A has a wall portion (tube wall) of a multilayer structure (laminate) that includes an inner layer 25, an outer layer 26, and a reinforcement member (reinforcement layer) 27 disposed between the inner layer 25 and the outer layer 26.

The material constituting the inner layer 25 is not specifically restricted. It is preferable, however, that a portion of the inner layer 25 that makes contact with a rectilinear stylet 3 or a curved stylet 4A at the time of inserting such a stylet into a lumen 211 is formed of a low-friction material. This helps ensure that each stylet can be moved longitudinally relative to the catheter body 21 under a comparatively small sliding resistance, thereby contributing to enhancement of operability. Examples of the low-friction material include various resin materials such as polyamides, polyether-polyamides, polyester-polyamides, polyesters (e.g., polyethylene terephthalate, polybutylene terephthalate, polyethylene naphthalate, etc.), polyurethane, flexible polyvinyl chloride, ABS resin, AS resin, and fluoro-resins such as polytetrafluoroethylene.

The material constituting the outer layer 26 is not particularly limited. Examples of the material applicable here for the outer layer 26 include various thermoplastic elastomers based on styrene, polyolefin, polyurethane, polyester, polyamide, polybutadiene, trans-polyisoprene, fluoro-rubber, chlorinated polyethylene or the like, and, further, combinations (polymer alloys, polymer blends, laminates and the like) of two or more of these thermoplastic elastomers.

The reinforcement member 27 has an extension/contraction-preventive function for preventing the catheter body 21 from extending or contracting in the longitudinal direction. This makes it possible to securely prevent an increase in frictional resistance between the catheter body 21 and each stylet which might arise from a decrease in diameter of the catheter body 21 according to a longitudinal extension of the catheter body 21.

The reinforcement member 27 is composed of a coil obtained by flattening a stainless steel wire rod and spirally winding the flattened product. This enables an extension/contraction-preventive function to be displayed rather assuredly.

As shown in FIG. 14(a), the winding density of the reinforcement member 27 is lower in the distal side than on the proximal side. That is, the windings per unit length of the reinforcement member 27 in the distal side is less than the windings per unit length of the reinforcement member 27 in the proximal side. As a result, the reinforcement member 27 is formed with a coarsely wound section (a section having a lower winding density) 271 and a densely wound section (a section having a higher winding density) 272. In a second assembled state, the catheter 2A is formed with a deformation section 24 at a portion where the coarsely wound section 271 is located. The coarsely wound section 271 is richer in flexibility (i.e., more flexible) than the densely wound section 272, which is preferable for the formation of the deformation section 24.

The winding density of the reinforcement member 27 may vary stepwise or continuously (gradually).

In addition, while the reinforcement member 27 is a spiral-shaped reinforcement member in the configuration shown in FIG. 14, the reinforcement member is not limited in this way. For example, the reinforcement member 27 may be a meshed reinforcement member.

As shown in FIG. 14(a) and FIG. 15, a main body section 231 of a hub 23 of the catheter 2A is formed at a proximal portion thereof with a deficit portion 233 where a wall portion is partly lost. That is, the deficit portion 233 at the proximal portion of the hub 23 is a cut-out portion.

On the other hand, the curved stylet 4A is provided with an engaging portion 45 which extends proximally further from a rectilinear section 44 and which engages the deficit portion 233 in the second assembled state. The engaging portion 45 is obtained by a method in which a linear body constituting the curved stylet 4A is wound into a ring-like shape. Of the curved stylet 4A, the engaging portion 45 engages the deficit portion 233 in the second assembled state, whereby rotation of the catheter 2A about its axis is restricted (see FIG. 15). This helps ensure that, at the time of forming the expanded portion 301 in the biological tissue 300, it is possible to assuredly deform the biological tissue 300 in a desired direction by pulling. Therefore, the expanded portion 301 can be formed in an assured manner.

Thus, in the catheter assembly 1 in this embodiment, the deficit portion 233 of the catheter 2A and the engaging portion 45 of the curved stylet 4A function as restriction means for restricting the curved stylet 4A from rotating about the axis of the catheter 2A in the second assembled state.

In addition, the outer surface of the curved stylet 4A has undergone a friction-reducing treatment for reducing the friction between the outer surface and the catheter 2A at the time of obtaining the second assembled state. This helps ensure that insertion of the curved stylet 4A into the catheter 2A can be carried out rather smoothly.

In the configuration shown in FIG. 14(b), the friction-reducing treatment consists in forming a coating film by application of a low-friction material. The low-friction material is not specifically restricted; for example, the same materials as those for the inner layer 25 can be used as the low-friction material.

### Seventh Embodiment

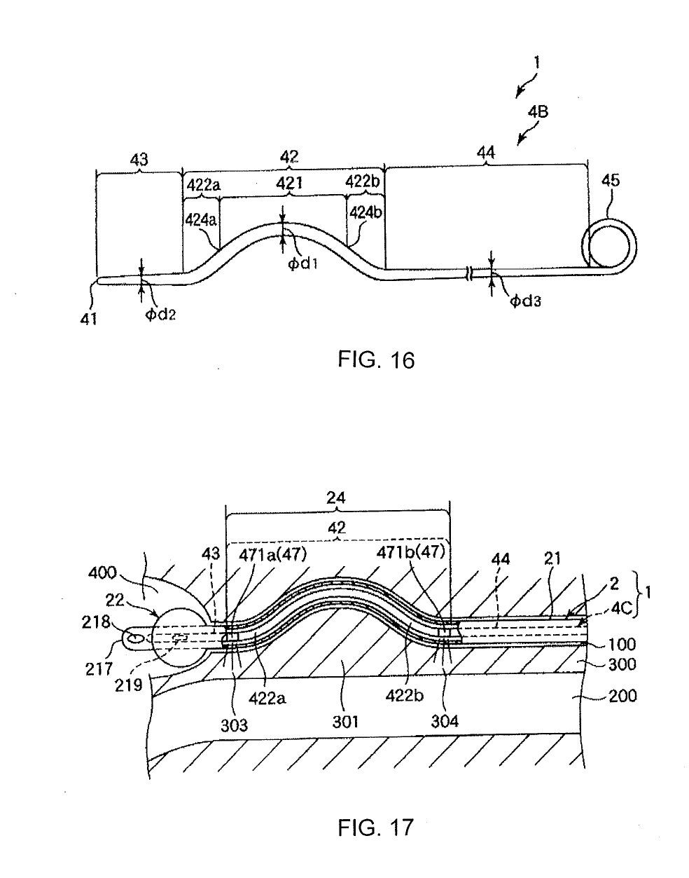

FIG. 16 illustrates a stylet in a catheter assembly according to a seventh embodiment. The following description focuses primarily on differences between this embodiment and the embodiments described above. Features in this embodiment that are similar to features in the embodiments described above are identified by common reference numerals and a detailed description of such features is not repeated.

This embodiment is the same as the second embodiment above, except for a difference in the configuration of the stylet.

As shown in FIG. 16, in a curved stylet 4B in this embodiment, the curved section 42 is greater in outer diametrical size (thicker or greater outer diameter) than a rectilinear section 43 disposed distally of the curved section 42, and is greater in outer diametrical size (thicker or greater outer diameter) than a rectilinear section 44 and an engaging portion 45 which are disposed proximally of the curved section 42. In other words, in the curved stylet 4B, the outside diameter φd₁ is greater than both of the outside diameter φd₂ of the rectilinear section 43 and the outside diameter φd₃ of the continuous part including the rectilinear section 44 and the engaging portion 45. This helps ensure that the curved section 42 is higher in rigidity than the other sections. Therefore, the curved stylet 4B is securely prevented from being unwillingly deformed by the catheter 2A in which it is inserted in the second assembled state, and so the curved state can be better maintained.

The rectilinear section 43 possesses a tapered shape wherein the outside diameter φd₂ of the rectilinear section 43 gradually decreases along the distal direction. This enables the curved stylet 4B to move in the longitudinal direction relative to a catheter body 21 under a comparatively small sliding resistance between the curved stylet 4B and the catheter body 21, which contributes to enhancement of operability. The operability is enhanced especially for a patient having a urethral lumen 100 that is originally tortuous.

### Eighth Embodiment

FIG. 17 illustrates a catheter assembly according to an eighth embodiment while the catheter assembly is in use. The following description focuses primarily on differences between this embodiment and the embodiments described above. Features in this eighth embodiment that are similar to features in the embodiments described above are identified by common reference numerals and a detailed description of such features is not repeated.

This embodiment is the same as the first embodiment, except for a difference in the configuration of the curved stylet.

As shown in FIG. 17, in this embodiment, a curved stylet 4C has a marker 47 for grasping the range of formation of the deformation section 24 in a second assembled state. That is, the marker 47 identifies the proximal and distal ends of the deformation section 24. The marker 47 is composed of a pair of luminous elements 471a and 471b which emit light. The luminous element 471a is disposed immediately on the distal side of a second portion 422a of the deformation section 24, while the luminous element 471b is disposed immediately on the proximal side of a second portion 422b of the deformation section 24. The luminous elements 471a and 471b are light emitting diodes, which are electrically connected to a power supply through a cable located in and passing through the curved stylet 4C.

Upon visual inspection of a biological tissue 300 from the side of a vaginal cavity 200, the light emitted from the luminous element 471a and transmitted through a distal portion 303 of the expanded portion 301 is visually confirmed. Also, the light emitted from the luminous element 471b and transmitted through a proximal portion 304 of the expanded portion 301 is visually confirmed. This makes it possible to rather easily grasp the portion of the biological tissue 300 at which is located the expanded portion 301. Consequently, a surgical treatment of the expanded portion 301 can be carried out in an assured manner.

### Ninth Embodiment

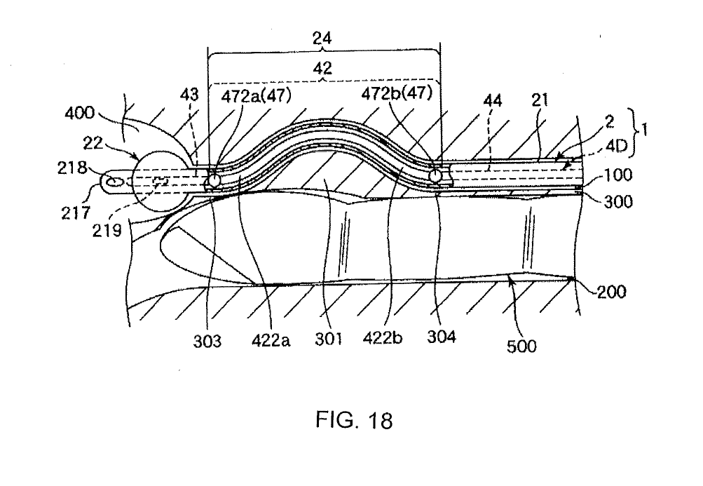

FIG. 18 illustrates a catheter assembly according to a ninth embodiment while the catheter assembly is in use. The following description focuses primarily on differences between this embodiment and the embodiments described above. Features in this ninth embodiment that are similar to features in the embodiments described above are identified by common reference numerals and a detailed description of such features is not repeated.

This embodiment is the same as the eighth embodiment described above, except for a difference in the configuration of marker.

As shown in FIG. 18, in this embodiment, a marker 47 of a curved stylet 4D is composed of projecting (protuberant) portions 472a and 472b possessing a spherical shape and projecting from the outer circumferential portion of the curved stylet 4D. The projecting portion 472a is located immediately on the distal side of a second portion 422a of a deformation section 24, while the projecting portion 472b is located immediately on the proximal side of a second portion 422b of the deformation section 24.

Upon insertion of a finger 500 into a vaginal cavity 200, the feeling of the projecting portion 472a is transmitted through a distal portion 303 of the expanded portion 301. That is, an individual can feel the projecting portion 472a. Similarly, the feeling of the projecting portion 472b is transmitted through a proximal portion 304 of the expanded portion 301 and so an individual can feel the projecting portion 472b. This makes it possible to rather easily grasp the portion of the biological tissue 300 at which the expanded portion 301 is formed or located. Consequently, surgical treatment of the expanded portion 301 can be performed in a reliable manner.

### Tenth Embodiment

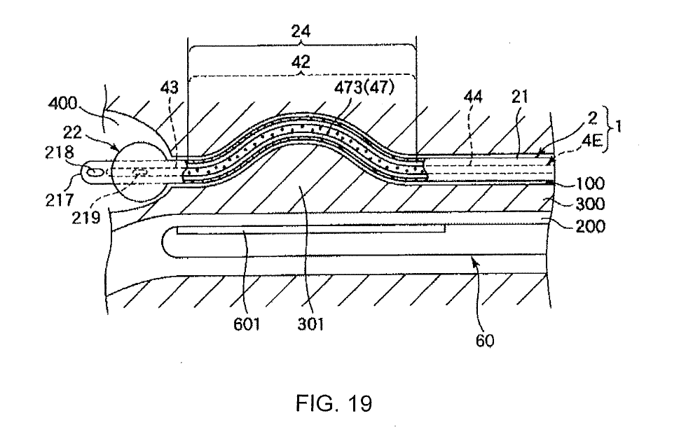

FIG. 19 illustrates a catheter assembly according to a tenth embodiment while the catheter assembly is in use. The following description focuses primarily on differences between this embodiment and the embodiments described above. Features in this tenth embodiment that are similar to features in the embodiments described above are identified by common reference numerals and a detailed description of such features is not repeated.

This embodiment is the same as the eighth embodiment above, except for a difference in the configuration of the marker.

The marker 47 of a curved stylet 4E in the FIG. 19 embodiment is composed of an imaging portion 473 having an imaging property allowing it to be recognized in an ultrasonic image obtained based on ultrasonic waves transmitted and received by an ultrasonic probe 60. The imaging portion 473 is comprised of minute projections and recesses which are formed throughout a curved section 42. The method for forming the projections and recesses is not particularly limited. Examples of the applicable method include a method in which roughening (blasting) is applied to the curved section 42, and a method in which a metallic powder is made to be supported on the curved section 42.

The ultrasonic probe 60, in the configuration shown in FIG. 19, is a long (elongated) instrument having an ultrasonic transducer 601 for transmission and reception of ultrasonic waves. The ultrasonic transducer 601 has, for example, a structure wherein electrodes are formed on both surfaces of a piezoelectric element composed of PZT (lead zirconate titanate).

In the ultrasonic transducer 601 of the ultrasonic probe 60, an ultrasonic wave is emitted and a reflected ultrasonic wave is received. Thus, emission and reception of ultrasonic waves are conducted. By the emission and reception of ultrasonic waves, an image of a part to be observed can be picked up. Specifically, in the ultrasonic transducer 601, the reflected ultrasonic wave received is converted into a signal, which is transmitted to a controller. The controller receives the signal, performs processing such as coordinate transformation, and generates an image signal representing the part to be observed. The image signal is sent from the controller to a display, on which the image of the part to be observed (namely, an ultrasonic image) is displayed. Generally speaking, the ultrasonic image is formed as follows. Based on the time from transmission of the ultrasonic wave from the ultrasonic transducer until the return of the reflected wave to the ultrasonic transducer, the distance to an object and the like are determined, and the object is visualized on the basis of determination results.

When the ultrasonic probe 60 is actuated in the state of the catheter 1 being inserted in the urethral lumen 100, an ultrasonic image of the imaging portion 473 is obtained. This makes it possible to rather easily grasp the part of the biological tissue 300 at which the expanded portion 301 is located. Consequently, a surgical treatment of the expanded portion 301 can be carried out in an assured manner.

### Eleventh Embodiment

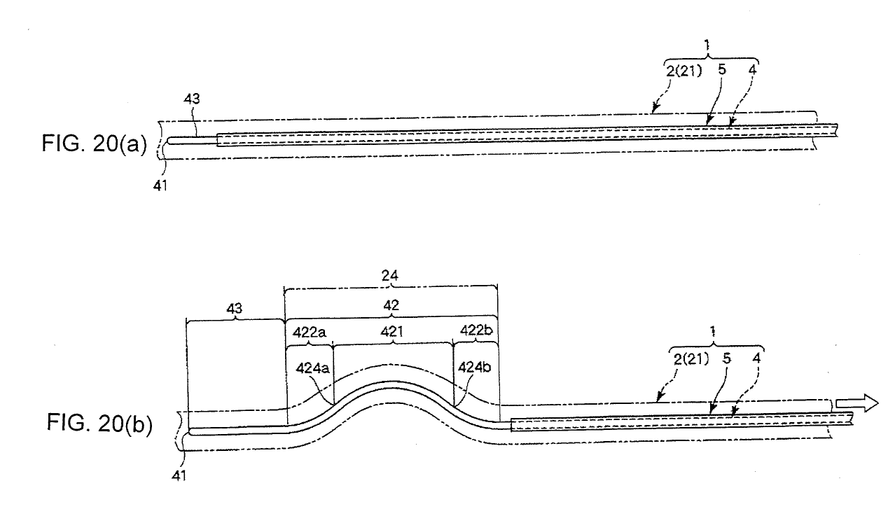

FIG. 20 illustrates a catheter assembly according to an eleventh embodiment while the catheter assembly is in use. The following description focuses primarily on differences between this embodiment and the embodiments described above. Features in this eleventh embodiment that are similar to features in the embodiments described above are identified by common reference numerals and a detailed description of such features is not repeated.

This embodiment is the same as the first embodiment above, except that the catheter assembly includes the correcting pipe.

As shown in FIGS. 20(a) and 20(b), in this embodiment, the catheter assembly 1 has a structure in which a rectilinear stylet 3 is omitted, and a correcting pipe 5 is provided in place of the rectilinear stylet 3. The correcting pipe 5 possesses a rigidity higher than both the catheter body 21 of the catheter 2 and the curved stylet 4, and is composed of a stainless steel pipe, for example. The correcting pipe 5 is rectilinear in shape, and has an outside diameter that is constant along the longitudinal direction or extent of the correcting pipe 5.

In addition, the curved stylet 4 can be inserted into and pulled out of the correcting pipe 5. In an insertion state in which the curved stylet 4 is located in the correcting pipe 5, a curved section 42 is forcibly deformed into a rectilinear shape (see FIG. 20(a)). On the other hand, when the correcting pipe 5 is retracted proximally from the curved section 42, the curved section 42 is released from a corrected state attained by virtue of the correcting pipe 5, to be restored into a curved shape (see FIG. 20(b)).

When a structure wherein the curved stylet 4 is inserted in the correcting pipe 5 is inserted into a lumen 211 of the catheter 2, as shown in FIG. 20(a), the catheter body 21 is formed into a rectilinear shape in conformity with the shape of the correcting pipe 5.

When the correcting pipe 5 is pulled out from the state shown in FIG. 20(a), the curved section 42 is restored into the curved shape as described above, as shown in FIG. 20(b), so that a deformation section 24 shaped along the shape of the curved section 42 is formed in the catheter body 21. By this deformation section 24, an expanded portion 301 is formed in a biological tissue 300, resulting in that a surgical treatment can be applied to the expanded portion 301.

### Twelfth Embodiment

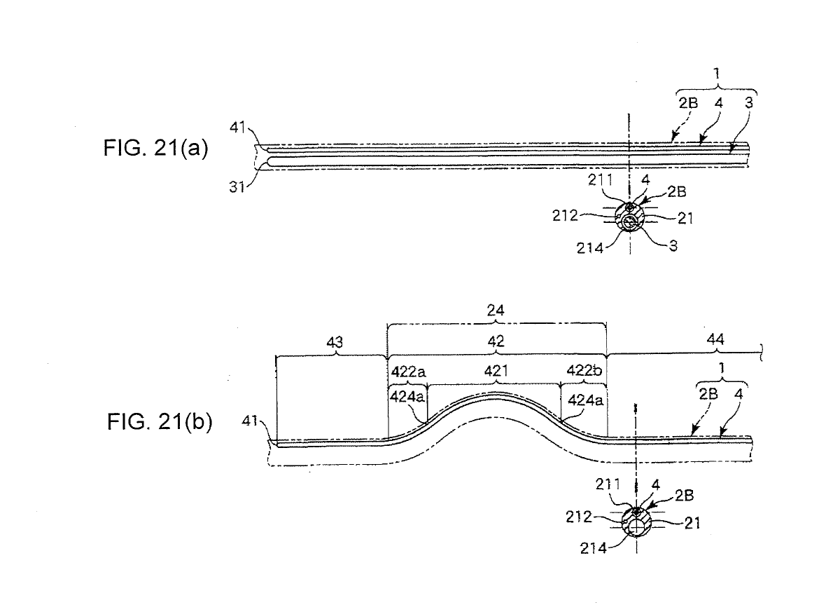

FIG. 21 illustrates a catheter assembly according to a twelfth embodiment while the catheter assembly is in use. The following description focuses primarily on differences between this embodiment and the embodiments described above. Features in this twelfth embodiment that are similar to features in the embodiments described above are identified by common reference numerals and a detailed description of such features is not repeated.

This embodiment is the same as the first embodiment above, except for a difference in the configuration of the catheter.

As shown in FIG. 21, in this embodiment, a catheter body 21 of a catheter 2B further has a lumen 214 in addition to lumens 211 and 212. A curved stylet 4 is positioned in the lumen 211, and a rectilinear stylet 3 (high-rigidity stylet) is positioned in the lumen 214.

In this embodiment, the curved stylet 4 is formed from a superelastic alloy. The rectilinear stylet 3 is greater in diametrical size (thicker or greater outer diameter) than the curved stylet 4, and is formed from a high-rigidity material, for example, stainless steel.

When the rectilinear stylet 3 is further inserted into the catheter 2B in an assembled state in which the curved stylet 4 is located in the catheter 2B, as shown in FIG. 21(a), a curved section 42 is forcibly deformed into a rectilinear shape along the rectilinear stylet 3. In this instance, a catheter body 21 is also formed into a rectilinear shape after the rectilinear stylet 3.

When the rectilinear stylet 3 is pulled out from the state shown in FIG. 21(a), the curved section 42 is released from the corrected state attained by virtue of the rectilinear stylet 3, and is restored to a curved state, as shown in FIG. 21(b). This results in that a deformation section 24 shaped after the shape of the curved section 42 is formed in the catheter body 21. By the deformation section 24, an expanded portion 301 is formed in a biological tissue 300, resulting in that a surgical treatment can be applied to the expanded portion 301.

### Thirteenth Embodiment

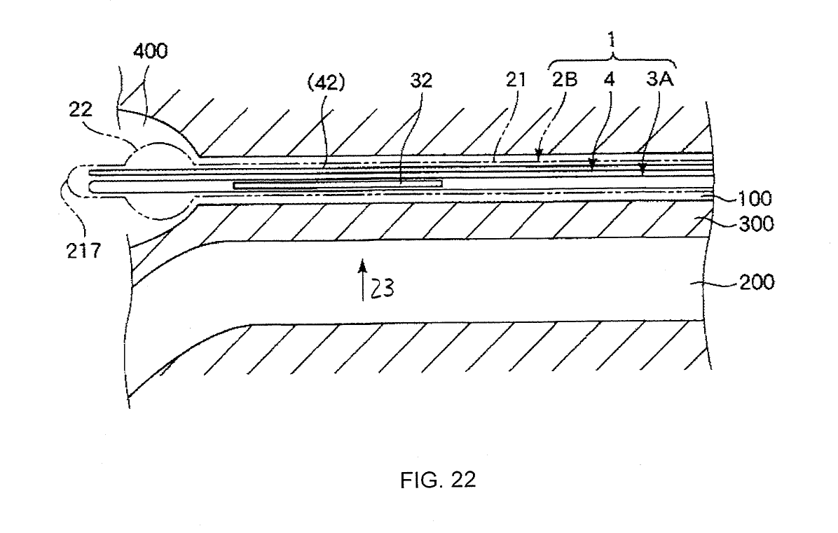

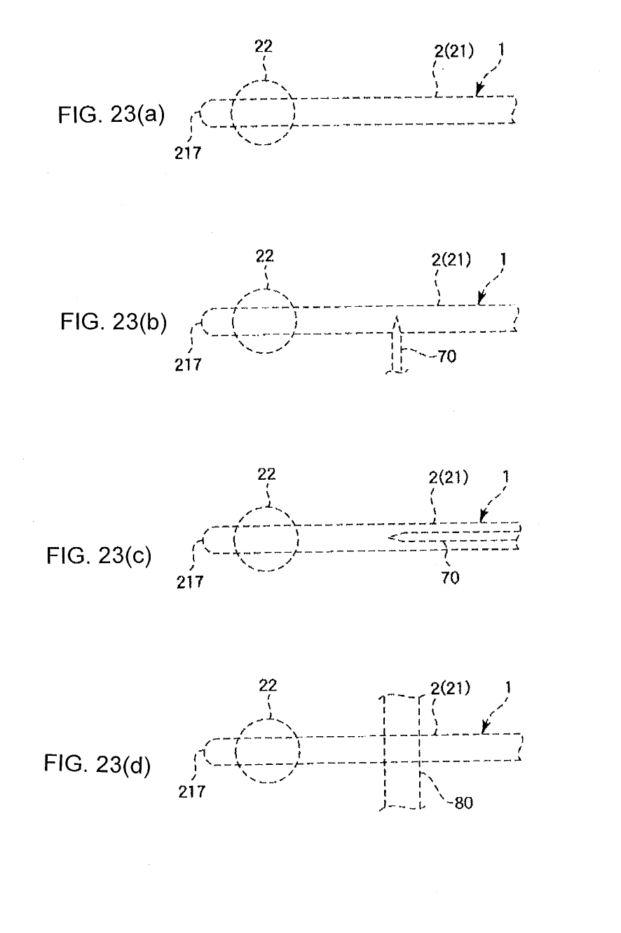

FIGS. 22 and 23 illustrate a catheter assembly according to a thirteenth embodiment while the catheter assembly is in use. The following description focuses primarily on differences between this embodiment and the embodiments described above. Features in this thirteenth embodiment that are similar to features in the embodiments described above are identified by common reference numerals and a detailed description of such features is not repeated.

This embodiment is the same as the twelfth embodiment above, except for a difference in the configuration of the high-rigidity stylet.

As shown in FIG. 22, in this embodiment, a rectilinear stylet 3A has a luminous element 32 that emits light. The luminous element 32 is disposed in a portion of the rectilinear stylet 3A that corresponds to (faces) a curved section 42 corrected into a rectilinear shape in a state attained upon insertion of the rectilinear stylet 3 further into a catheter 2B in an assembled state in which a curved stylet 4 is inserted in the catheter 2B. The luminous element 32 is a light emitting diode, which is electrically connected to a power supply through a cable located in and passing though the rectilinear stylet 3A.

When the state shown in FIG. 22 is visually inspected from the side of a vaginal cavity 200 (in the direction of arrow 23 in FIG. 22), the light emitted from the luminous element 32 and transmitted through a biological tissue 300 can be confirmed. This ensures that the biological tissue 300 in one of the states illustrated in FIG. 23(a)-23(d), for example, is observed.

In the state shown in FIG. 23(a), surgical treatment has not yet been applied to the biological tissue 300. In the state shown in FIG. 23(b), a needle body 70 has punctured the biological tissue 300, in a direction orthogonal to the longitudinal direction of the urethral lumen 100. In the state shown in FIG. 23(c), the needle body 70 has punctured the biological tissue 300 in a direction parallel to the longitudinal direction of the urethral lumen 100. In the state shown in FIG. 23(d), a belt 80 as an implant is set indwelling in the biological tissue 300.

In this way, it is possible to grasp in what state the biological tissue 300 is when observed.

### Fourteenth Embodiment

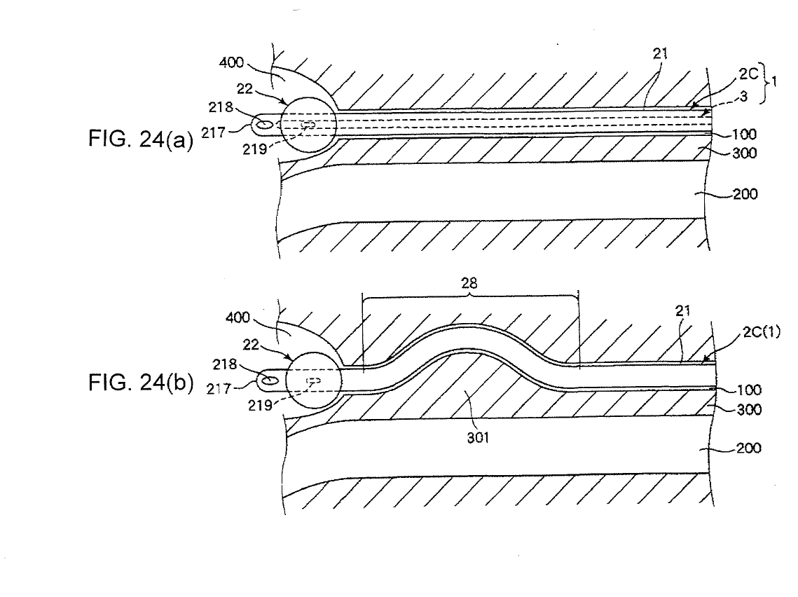

FIGS. 24(a) and 24(b) illustrate a catheter assembly according to a fourteenth embodiment while the catheter assembly is in use. The following description focuses primarily on differences between this embodiment and the embodiments described above. Features in this fourteenth embodiment that are similar to features in the embodiments described above are identified by common reference numerals and a detailed description of such features is not repeated.

This embodiment is the same as the first embodiment above, except for a difference in the configuration of the catheter.

As shown in FIGS. 24(a) and 24(b), in this embodiment, the catheter assembly 1 includes a catheter 2C and a rectilinear stylet 3, with a curved stylet 4 being omitted.

A catheter body 21 of the catheter 2C is provided, at its intermediate portion in the longitudinal direction, specifically, at its portion on the distal side, with a curved section 28 that is curved in a natural state, namely, in a state where the rectilinear stylet 3 is not inserted therein and when no force (external or internal) is applied to the catheter body. The curved shape of the curved section 28 is the same as that of the curved section 42 of the curved stylet 4. The curved section 28 is higher in rigidity than the rectilinear stylet 3. In order that the curved section 28 has such a rigidity, it suffices, for example, to provide a structure wherein a reinforcement member composed of stainless steel is disposed.

As shown in FIG. 24(a), in an assembled state in which the rectilinear stylet 3 is inserted in the catheter 3, the curved section 28 is forcibly deformed into a rectilinear shape along the rectilinear stylet 3. This helps ensure that at the time when the catheter assembly 1 in the assembled state is inserted into a urethral lumen 100, the catheter body 21 can be prevented from being unwillingly curved or bent during the insertion process. Therefore, the inserting operation can be carried out easily and assuredly.

As shown in FIG. 24(b), in a state wherein the rectilinear stylet 3 has been pulled out of the catheter 2C, the curved section 28 can pull a portion of a biological tissue 300 that faces the curved section 28, toward the curved section 28 side (the upper side in FIG. 24(b)). This results in that the portion is spread in direction for spacing the urethral lumen 100 and a vaginal cavity 200 away from each other, to be an expanded portion 301. Then, at the time of applying a surgical treatment to the expanded portion 301, the treatment can be carried out more easily and securely.

### Fifteenth Embodiment

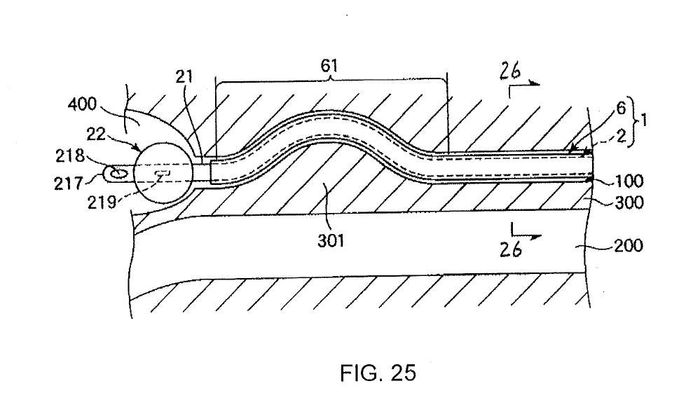

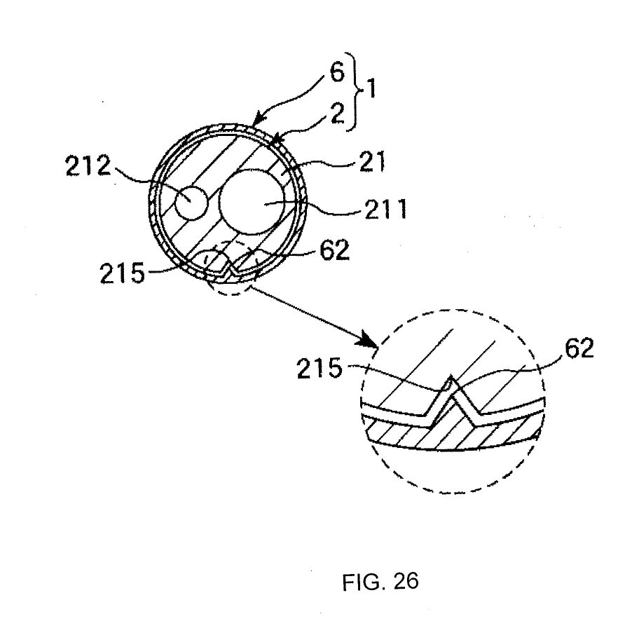

FIGS. 25 and 26 illustrate a catheter assembly according to a fifteenth embodiment while the catheter assembly is in use. The following description focuses primarily on differences between this embodiment and the embodiments described above. Features in this fifteenth embodiment that are similar to features in the embodiments described above are identified by common reference numerals and a detailed description of such features is not repeated.

This embodiment is the same as the first embodiment above, except that the catheter assembly includes a hard pipe.

As shown in FIGS. 25 and 26, in this embodiment, the catheter assembly 1 has a configuration in which a curved stylet 4 is omitted and a long (elongated) hard pipe 6, into which a catheter body 21 can be inserted and from which the catheter body 21 can be pulled out, is provided in place of the curved stylet 4. The hard pipe 6 is higher in rigidity than the catheter body 21 of a catheter 2, and is composed of, for example, a stainless steel tube shaped body. The hard pipe 6 has an inside diameter and an outside diameter which are constant along the longitudinal direction or longitudinal extent of the hard pipe 6.

In addition, the hard pipe 6 has a curved section 61 at its intermediate portion in the longitudinal direction, specifically at its portion on the distal side. The curved shape of the curved section 61 is the same as that of a curved section 42 of a curved stylet 4. In an assembled state attained by inserting the catheter body 21 into the hard pipe 6, a portion of the catheter body 21 that is located in the curved section 61 is shaped (deformed) to match the shape of the curved section 61.

When the hard pipe 6 in the assembled state is inserted in a urethral lumen 100, the curved section 61 pulls a portion of a biological tissue 300 that faces the curved section 61 toward the curved section 61 side (the upper side in FIG. 25), as shown in FIG. 25. This results in that the portion is spread, in a direction for spacing the urethral lumen 100 and a vaginal cavity 200 away from each other, to be an expanded portion 301. Then, at the time of applying a surgical treatment to the expanded portion 301, the treatment can be carried out rather easily and assuredly.

As shown in FIG. 26, a projecting rib 62 is formed at an inner circumferential portion of the hard pipe 6. This rib 62 extends along the longitudinal direction of the hard pipe 6 and projects radially inwardly in the illustrated embodiment. On the other hand, the outer circumferential portion of the catheter body 21 is formed with a groove 215 configured to receive the rib 62. The rib 62 is inserted into the groove 215, whereby rotation of the hard pipe 6 about the axis of the hard pipe is restricted. This helps ensure that, at the time of forming the expanded portion 301 in the biological tissue 300, the biological tissue 300 can be securely deformed in directions in which the biological tissue 300 is to be deformed by pulling. Therefore, the expanded portion 301 can be formed in an assured manner.

### Sixteenth Embodiment

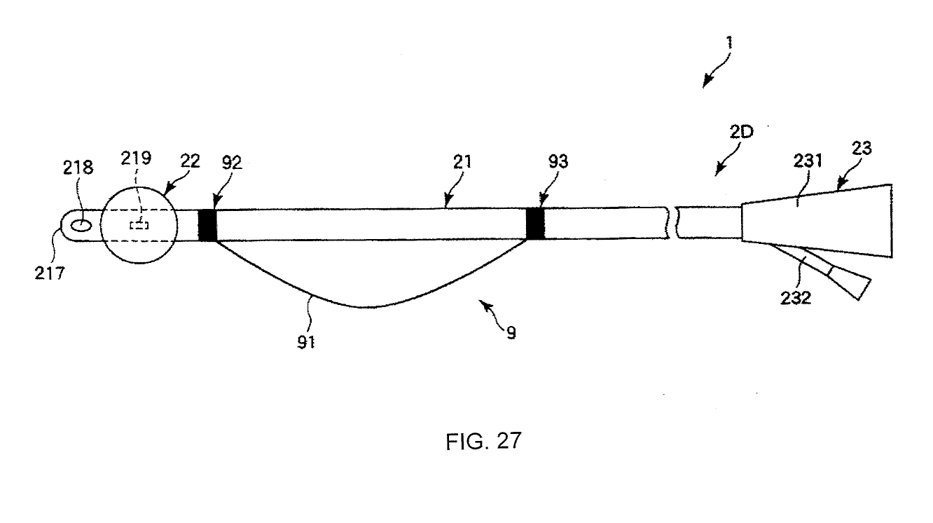

FIGS. 27-29 illustrate a catheter assembly according to an sixteenth embodiment while the catheter assembly is in use. The following description focuses primarily on differences between this embodiment and the embodiments described above. Features in this sixteenth embodiment that are similar to features in the embodiments described above are identified by common reference numerals and a detailed description of such features is not repeated.

This embodiment is the same as the first embodiment above, except for a difference in the configuration of the catheter.

As shown in FIG. 27, in the catheter assembly 1 in this embodiment, a catheter 2D has a radiopaque section 9 which is disposed on the distal side with respect to a catheter body 21 and extends in the longitudinal direction of the catheter body 21. The radiopaque section 9 is formed from radiopaque material. The radiopaque material is not specifically restricted. An example of the material includes tungsten. This helps ensure that the radiopaque section 9 can follow up to variations in the urethra shape attendant on curving of the catheter 2, so that variations in the urethra shape can be confirmed through a radioscopic image.

The radiopaque section 9 includes: a linear portion 91 which is linear in shape and is flexible, and a first fixing portion 92 and a second fixing portion 93 by which both end parts of the linear portion 91 are supported on and fixed to the catheter body 21.

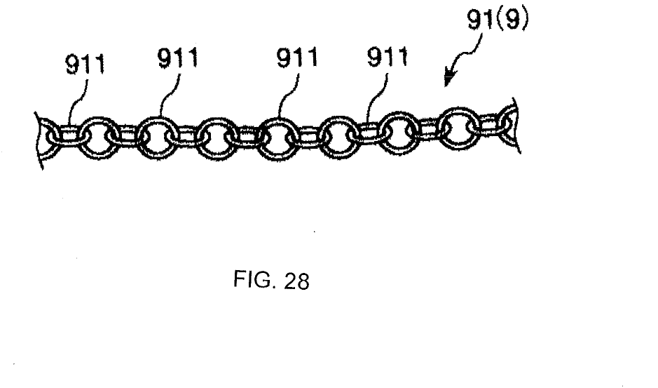

As shown in FIG. 28, the linear portion 91 has a chain-like structure in which a multiplicity of circular ring bodies 911 are linked along the catheter longitudinal direction. This helps ensure that the linear portion 91 securely has flexibility. Therefore, when the catheter 2D is inserted in a urethral lumen 100, the linear portion 91 can assuredly follow the curvature of the urethral lumen 100. In addition, the linear portion 91 having the chain structure helps ensure that the area of contact between the linear portion 91 and the outer circumferential surface of the catheter body 21 can be reduced to a comparatively small level. Consequently, adhesion of the linear portion 91 onto the outer circumferential surface of the catheter body 21 can be inhibited or prevented.

The circular ring bodies 911 preferably have the same diameter.

In addition, while the linear section 91 has the structure in which the circular ring bodies 911 are mutually linked in the aforementioned case, this configuration is not restrictive. For instance, the linear section 91 may have a structure in which polygonal ring bodies are mutually linked, or a structure in which spherical bodies are mutually linked.

The first fixing portion 92 is a band supporting a distal part of the linear portion 91, and the second fixing portion 93 is a band supporting a proximal part of the linear portion 91. The spacing between the first fixing portion 92 and the second fixing portion 93 can be changed. This makes it possible to regulate the degree of flexure of the linear portion 91.

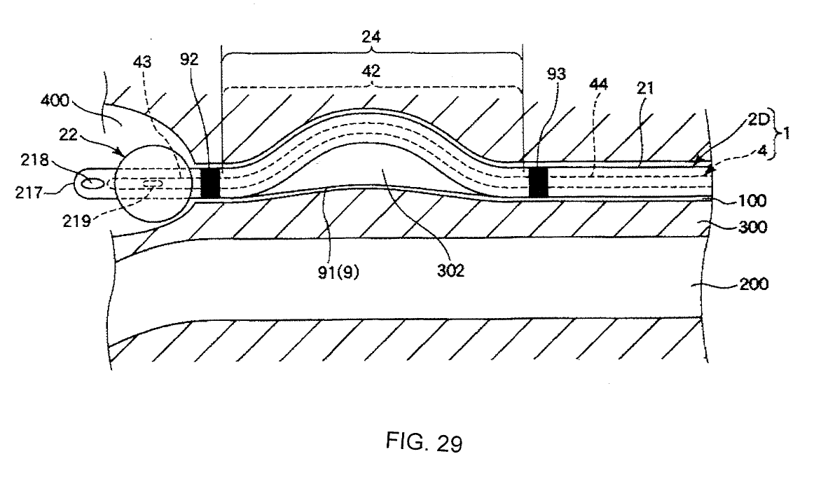

When the catheter assembly 1 is disposed in a urethral lumen 100, as shown in FIG. 29, depending on the patient (the case) there may be cases where, even though a portion of a biological tissue 300 that faces a deformation section 24 is pulled by the deformation section 24, the urethral lumen 100 is expanded to cause a space 302 to be further formed within the urethral lumen 100, resulting in the expanded portion 301 (refer to FIG. 25) of the biological tissue 300 not being formed. However, when the catheter assembly 1 according to this embodiment is used, the radiopaque section 9 (the linear portion 91) capable of flexibly following up to the deformation of the urethral lumen 100 can be confirmed through the radioscopic image, whereby it is possible to rather easily check whether or not the expanded portion 301 has been formed. Consequently, it is possible to relatively easily judge whether or not a surgical treatment of the expanded portion 301 is to be carried out.

In this embodiment, the curved stylet 4 shown in FIG. 20 can be combined with the catheter 2D shown in FIG. 27.

### Seventeenth Embodiment

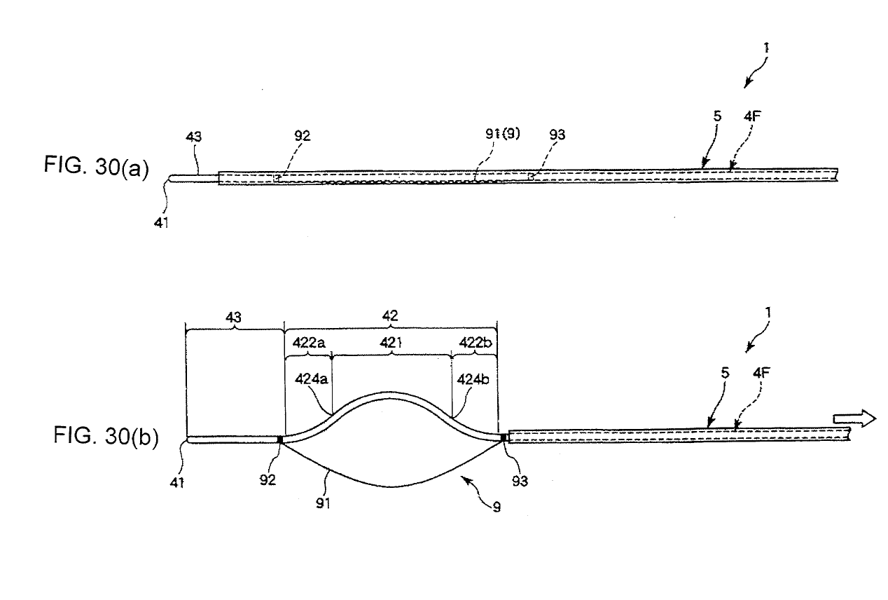

FIGS. 30(a) and 30(b) illustrate a catheter assembly according to a seventeenth embodiment while the catheter assembly is in use. The following description focuses primarily on differences between this embodiment and the embodiments described above. Features in this seventeenth embodiment that are similar to features in the embodiments described above are identified by common reference numerals and a detailed description of such features is not repeated.

This embodiment is the same as the eleventh embodiment above, except for a difference in the configuration of the stylet.

As shown in FIGS. 30(a) and 30(b), in the catheter assembly 1 according to this embodiment, a curved stylet 4F has a radiopaque section 9 which is disposed on the distal side with respect to the curved stylet 4F and extends in the longitudinal direction of the curved stylet 4F. The radiopaque section 9 is formed from a radiopaque material, which is not specifically restricted. An example of the material includes tungsten. This helps ensure that the radiopaque section 9 can follow up to variations in the urethra shape attendant on curving of the curved stylet 4F, so that variations in the urethra shape can be confirmed through a radioscopic image.

The radiopaque section 9 includes a linear portion 91 which is linear and flexible, and a first fixing portion 92 and a second fixing portion 93 by which both end parts of the linear portion 91 are supported and fixed. The respective configurations of the linear portion 91, the first fixing portion 92 and the second fixing portion 93 are the same as the respective configurations of the linear portion 91, the first fixing portion 92 and the second fixing portion 93 of the radiopaque section 9 described in the sixteenth embodiment above and shown in FIGS. 27-29.

The radiopaque section 9 with such a configuration is disposed in the same position as a curved section 42 of the curved stylet 4F, in the longitudinal direction of the curved stylet 4F.

At the time of insertion into a urethral lumen 100 of the catheter assembly 1 in an insertion state in which the curved stylet 4F is inserted into a correcting pipe 5 up to the curved section 42 (the radiopaque section 9), as shown in FIG. 30(a), sliding resistance due to the radiopaque section 9 can be prevented from increasing, and the catheter body 1 can be rather easily inserted up to a desired position. In addition, when the correcting pipe 5 is pulled in the proximal direction, as shown in FIG. 30(b), the curved section 42 (the radiopaque section 9) of the curved stylet 4F protrudes from the correcting pipe 5. In this instance, the curved section 42 is curved, and, simultaneously, the radiopaque section 9 undergoes flexure according to the degree of the curving.

Like in the sixteenth embodiment above, when the catheter assembly 1 is disposed in the urethral lumen 100, depending on the patient (the case) there may be cases where, even though a portion of a biological tissue 300 that faces a deformation section 24 is pulled by the deformation section 24, the urethral lumen 100 is expanded to cause a space 101 to be further formed within the urethral lumen 100, resulting in that an expanded portion 301 of the biological tissue 300 would not be formed (see FIG. 29). However, when the catheter assembly 1 according to this embodiment is used, the radiopaque section 9 (the linear portion 91) in the state shown in FIG. 30(b) that flexibly follows up to the deformation of the urethral lumen 100 can be confirmed through the radioscopic image, whereby it is possible to easily check whether or not the expanded portion 301 has been formed. As a result, it is possible to rather easily judge whether or not a surgical treatment of the expanded portion 301 is to be carried out.

### Eighteenth Embodiment

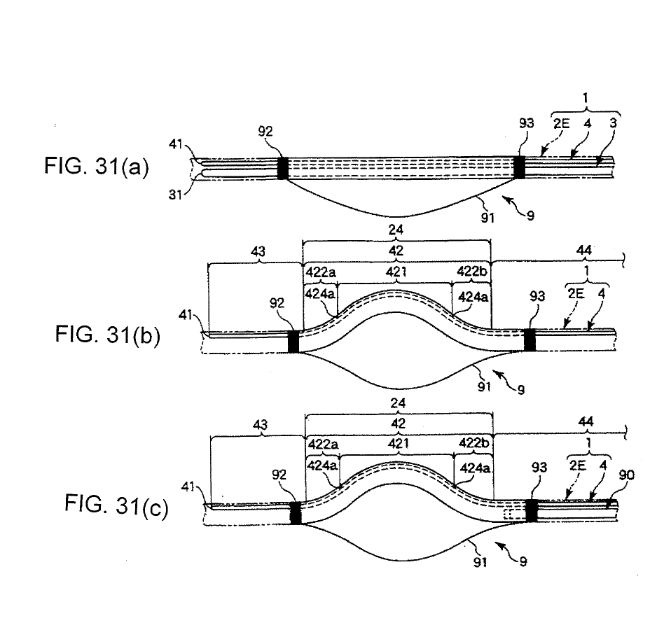

FIGS. 31(a)-(c) illustrate a catheter assembly according to an eighteenth embodiment while the catheter assembly is in use. The following description focuses primarily on differences between this embodiment and the embodiments described above. Features in this eighteenth embodiment that are similar to features in the embodiments described above are identified by common reference numerals and a detailed description of such features is not repeated.

This embodiment is the same as the twelfth embodiment above, except for a difference in the configuration of the catheter.

As shown in FIGS. 31(a)-(c), in the catheter assembly 1 according to this embodiment, a catheter 2E has a radiopaque section 9 which is disposed on the distal side with respect to a catheter body 21 and extends in the longitudinal direction of the catheter body 21. The radiopaque section 9 is formed from a radiopaque material, which is not particularly limited. An example of the material includes tungsten. This helps ensure that the radiopaque section 9 can follow up to variations in the urethra shape attendant on curving of the catheter 2E, so that variations in the urethra shape can be confirmed through a radioscopic image.

The radiopaque section 9 includes a linear portion 91 which is linear in shape and is flexible, and a first fixing portion 92 and a second fixing portion 93 by which both end parts of the linear portion 91 are supported and fixed. The respective configurations of the linear portion 91, the first fixing portion 92 and the second fixing portion 93 are the same as the respective configurations of the linear portion 91, the first fixing portion 92 and the second fixing portion 93 of the radiopaque section 9 described in the sixteenth embodiment above and shown in FIGS. 27-29.

The radiopaque section 9 with such a configuration is disposed in the same position as a curved section 42 of a curved stylet 4 in an assembled state, in the longitudinal direction of the catheter 2E.

In this embodiment, the catheter body 21 is composed of a transparent tube body, the material of which is not specifically restricted. An example of the material includes polyethylene. In addition, the catheter body 21 may not necessarily be entirely formed of a transparent material. Thus, only a part of the catheter body 21, with a deformation section 24 as a center of the part, may be composed of a transparent member.

The degree of flexure of the linear portion 9 of the radiopaque section 9 in a state shown in FIG. 31(a) where a rectilinear stylet 3 and a curved stylet 4 are inserted in the catheter 2E is different from that in a state shown in FIG. 31(b) where the rectilinear stylet 3 has been pulled out of the catheter 2E whereas the curved stylet 4 is left inserted. The linear portion 91 shows greater flexure in the state shown in FIG. 31(b) than in the state shown in FIG. 31(a).

In addition, as shown in FIG. 31(c), the catheter 2E permits an endoscope 90 to be inserted into the catheter after the rectilinear stylet 3, previously inserted in a lumen 214, is pulled out. The radiopaque section 9 follows up to variations in the urethra shape attendant on curving of the catheter 2B, and the variations in the urethra shape can be confirmed by use of the endoscope 90.

Meanwhile, like in the sixteenth embodiment above, when the catheter assembly 1 is disposed in a urethral lumen 100, depending on the patient (the case) there may be cases where, even though a portion of a biological tissue 300 that faces the deformation section 24 is pulled by the deformation section 24, the urethral lumen 100 is expanded to cause a space 101 to be further formed within the urethral lumen 100, resulting in that an expanded portion 301 of the biological tissue 300 would not be formed (see FIG. 29). When the catheter assembly 1 according to this embodiment is used, however, the radiopaque section 9 (the linear portion 91) in the state shown in FIGS. 31(b) and (c) that flexibly follows up to the deformation of the urethral lumen 100 can be confirmed through the radioscopic image, whereby it is possible to rather easily check whether or not the expanded portion 301 has been formed. As a result, it is possible to easily judge whether or not a surgical treatment of the expanded portion 301 is to be carried out.

While the catheter assembly disclosed here has been described above with reference to the embodiments shown in the drawings, the invention is not restricted to the illustrated and described embodiments. The sections or portions constituting the catheter assembly can be replaced by sections or portions of other configurations which can exhibit equivalent or similar functions. Features and structures may also be added.

In addition, the catheter assembly may be configured to include a combination of the configurations (features) of two or more of the above embodiments.

The catheter assembly disclosed here generally includes: a catheter provided with a catheter body having at least one lumen; and a stylet that includes a linear body of higher rigidity than the catheter body and positioned in the lumen, with the catheter assembly being configured to assume an assembled state when assembled by inserting the stylet into the lumen. The catheter assembly is configured so that: the stylet has a curved section, a part of which is curved; the catheter body is formed with a deformation section that has been deformed in a curved manner along the curved shape of the curved section, at a portion where the curved section is located in the assembled state; and the catheter is so configured that, when the catheter is inserted into one of two living-body lumens adjacent to each other with biological tissue therebetween in the assembled state, the deformation section curves a portion of the one living-body lumen that faces the deformation section. Therefore, when a surgical treatment is applied to the biological tissue between the two living-body lumens, the treatment can be carried out relatively easily and assuredly.

The detailed description above describes features and aspects of embodiments of a catheter assembly. The invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A catheter assembly comprising:
a catheter comprised of a catheter body possessing at least one lumen, the catheter body possessing a rigidity;
a stylet comprised of a linear body positionable in the lumen in the catheter body in an assembled state of the catheter assembly;
the stylet including a curved section that is curved;
the linear body of the stylet, inclusive of the curved section, possessing a rigidity greater than the rigidity of the catheter body;
the catheter body including a deformation section that is deformable so that when the stylet is positioned in the lumen with the curved section of the stylet located in the lumen in the assembled state of the catheter assembly the deformation section of the catheter body deforms to a curved shape caused by the curved section of the stylet, and when the stylet is removed from the lumen the deformation section of the catheter body changes to a shape different from the curved shape; and
when the catheter in the assembled state is positioned in one of two living-body lumens adjacent to each other with biological tissue between the two living-body lumens, the deformation section of the catheter body which possess the curved shape curves a portion of the one living-body lumen facing the deformation section.

2. The catheter assembly according to claim 1, wherein the curved section of the stylet includes a first curved portion curved in one direction, and two second curved portions each curved in an opposite direction compared with the first curved portion, one of the second curved portions being located immediately on a distal side of the first curved portion and the other second curved portion being located immediately on a proximal side of the first curved portion.

3. The catheter assembly according to claim 2, wherein the stylet includes an other section different from the curved section, the other section of the stylet possessing a rectilinear shape in a natural state when no force is applied to the stylet, and a distance between a straight line and a central portion of the first curved portion in a longitudinal direction of the first curved portion is not more than 10 mm, the straight line interconnecting both end sides of the stylet, and the curved section possessing a length along the straight line that is not more than four times the distance.

4. The catheter assembly according to any claim 1, wherein the curved section is provided at an intermediate portion of the stylet in a longitudinal direction of the stylet so that a portion of the stylet extends from a proximal end of the curved section to a proximal end of the stylet and so that a portion of the stylet extends from a distal end of the curved section to a distal end of the stylet, the stylet possessing an outer diametrical size throughout the curved section that is greater than the outer diametrical size throughout the portion of the stylet on the distal side of the curved section and throughout the portion of the stylet on the proximal side of the curved section.

5. The catheter assembly according to claim 1, wherein the curved section is located at an intermediate portion of the stylet relative to a longitudinal direction of the stylet, and a portion of the stylet on a distal side of the curved section possesses a tapered outer shape in which an outside diameter of the stylet gradually decreases along a distal direction.

6. The catheter assembly according to claim 1, further comprising a marker on the stylet identifying opposite ends of the curved section to permit identification of a range over which the deformation section extends.

7. The catheter assembly according to claim 1, wherein the stylet includes a portion configured to enable a user to understand a direction of curvature of the curved section.

8. The catheter assembly according to claim 1, including restriction means for restricting rotation of the stylet about an axis of the catheter body in the assembled state.

9. The catheter assembly according to claim 1, wherein the stylet possesses a reduced friction outer surface treated to reduce friction between the outer surface of the stylet and the catheter when inserting the stylet into the catheter so that the catheter assembly is in the assembled state.

10. The catheter assembly according to claim 1, wherein the catheter body includes a longitudinally extending reinforcement member that prevents extension and contraction of the catheter body in a longitudinal direction of the catheter body.

11. The catheter assembly according to claim 10, wherein the catheter body includes a longitudinally extending inner layer and a longitudinally extending outer layer, and the longitudinally extending reinforcement member is positioned between the longitudinally extending inner layer and the longitudinally extending outer layer, and the reinforcement member includes a spirally wound coil.

12. The catheter assembly according to claim 11, wherein the spirally wound coil possesses a winding density, the winding density of a distal portion of the spirally wound coil at a distal portion of the catheter body being less than the winding density of a proximal portion of the spirally wound coil at a proximal portion of the catheter body.

13. The catheter assembly according to claim 12, wherein the deformation section is located at the distal portion of the catheter body at which the winding density of the spirally wound coil is relatively lower compared to the proximal portion of the catheter body.

14. The catheter assembly according to claim 1, wherein the stylet is insertable into the lumen in the catheter body and is removable from the lumen in the catheter body, and further comprising a correcting pipe insertable into the lumen together with the stylet to forcibly deform the curved section into a rectilinear shape.

15. The catheter assembly according to claim 1, wherein the catheter body has two lumens, and the stylet is a first stylet insertable into one of the two lumens, and a further comprising a second high-rigidity stylet insertable into the other lumen and possessing higher rigidity than the rigidity of the first stylet to forcibly deform the curved section in the assembled state.

16. A catheter assembly comprising:
a catheter comprised of a catheter body, the catheter body including a lumen;
a stylet including a curved section that is curved in a natural state in which no force is applied to the curved section, the curved section of the stylet possessing a rigidity;
at least a portion of the catheter possessing a rigidity that is less than the rigidity of the curved section of the stylet;
the curved section of the stylet which is curved being positioned inside the lumen in the catheter body so that the portion of the catheter body which possesses a rigidity less than the rigidity of the curved section surrounds the curved section of the stylet and is curved in a manner matching the curvature of the curved section of the stylet so that the portion of the catheter body is a curved portion; and
the rigidity of the curved section of the stylet causing, when the catheter is positioned in one of two living-body lumens adjacent to each other with biological tissue between the two living-body lumens, the curved portion of the catheter body to spread a portion of the biological tissue between the two living-body lumens to space the two living-body lumens further away from each other as compared to before positioning the catheter in the one of two living-body lumens.

17. The catheter assembly according to claim 16, wherein the stylet possesses a distal-most end and a proximal-most end, the curved section of the stylet including a distal-most end and a proximal-most end, the distal-most end of the curved section being proximally spaced from the distal-most end of the stylet, and the proximal-most end of the curved section being distally spaced from the proximal-most end of the stylet.

18. A method comprising:
inserting a catheter into one of two living-body lumens positioned adjacent to each other with biological tissue between the two living-body lumens;
the insertion of the catheter into the one living-body lumen being performed to position at least a part of the catheter in the one living-body lumen; and
changing a shape of a portion of the catheter that is positioned in the one living-body lumen by curving the portion of the catheter in a direction away from the other living-body lumen so that the curving portion of the catheter causes a portion of the biological tissue facing the curving portion to be pulled away from the other living-body lumen.

19. The method according to claim 18, wherein the catheter inserted into the one living-body lumen is a rectilinear catheter, and the changing of the shape of the portion of the catheter that is positioned in the one living-body lumen comprises inserting a stylet that includes a curved section into the rectilinear catheter, the curved section of the stylet possessing a rigidity greater than a rigidity of the portion of the catheter so that the portion of the catheter assumes a curved shape matching the curved section.

20. The method according to claim 18, wherein a stylet is positioned inside the catheter before insertion of the catheter into the one living-body lumen, the stylet including a section which is curved in a natural state in which no force is applied to the curved section, the section of the stylet possessing a rigidity greater than a rigidity of a portion of the catheter overlying the section of the stylet, wherein the portion of the catheter and the section of the stylet are positioned inside a pipe which possesses a rigidity greater than the rigidity of the curved section of the stylet and greater than the rigidity of the portion of the catheter overlying the portion of the stylet; the changing of the shape of the portion of the catheter that is positioned in the one living-body lumen comprising moving the pipe relative to the stylet and the catheter to expose the portion of the catheter so that the curved section of the stylet curves to its natural state, and the curved section of the stylet causing the portion of the catheter overlying the curved section to assume a curved shape.
